(19) United States
(12) Patent Application Publication
(10) Pub. No.: US 2010/0291036 A1
(43) Pub. Date: Nov. 18, 2010

(54) COMPOSITIONS AND METHODS FOR MODULATING VASCULAR ENDOTHELIAL GROWTH FACTOR C (VEGF-C) EXPRESSION

(75) Inventors: Michal Neeman, Mazkeret Batya (IL); Batya Cohen, Tel Aviv (IL); Stav Sapoznik, Jerusalem (IL); Shifra Ben-Dor, Jerusalem (IL); Gila Meir, Rishon LeZion (IL)

Correspondence Address: FENNEMORE CRAIG, 3003 NORTH CENTRAL AVENUE, SUITE 2600, PHOENIX, AZ, 85012, US

(73) Assignee: YEDA RESEARCH AND DEVELOPMENT CO.LTD, at The Weizmann Institute of Science, Rehovot (IL)

(21) Appl. No.: 12/812,735

(22) PCT Filed: Jan. 25, 2009
(86) PCT No.: PCT/IL2009/000100

§ 371 Date: Jul. 13, 2010

Related U.S. Application Data
(60) Provisional application No. 61/023,105, filed on Jan. 24, 2008.

(51) Int. Cl. A61K 35/00; C07H 21/00; A61K 31/7088; A61K 38/18; A61P 35/00; A61P 37/02; A61P 29/00; A61P 31/00; A61P 17/02

(52) U.S. Cl. 424/93.2; 536/24.5; 514/44 A; 514/7.6; 514/44 R

(57) ABSTRACT

The present invention relates to compounds and methods for inhibiting the expression of vascular endothelial growth factor-C (VEGF-C) in a target cell. Particularly, the present invention relates to antisense polynucleotides complementary to a lens epithelium-derived growth factor (LEDGF/p75) mRNA and uses thereof for inhibiting tumor progression and tumor metastasis. The present invention further relates to uses of LEDGF/p75 polypeptide or a nucleic acid encoding same for treating endothelial cell related conditions, particularly inflammation and edema.

### FIELD OF THE INVENTION

The present invention relates to compositions and methods for inhibiting the expression of vascular endothelial growth factor-C (VEGF-C) in a target cell. Particularly, the present invention relates to antisense polynucleotides complementary to a lens epithelium-derived growth factor (LEDGF/p75) mRNA and uses thereof for inhibiting tumor progression and tumor metastasis. The present invention further relates to uses of LEDGF/p75 polypeptide or a nucleic acid encoding same for treating endothelial cell related conditions, particularly inflammation and edema.

### BACKGROUND OF THE INVENTION

Blood and lymphatic vessels provide complementary functions in maintenance of tissue homeostasis. Blood circulation is optimally designed for efficient delivery and clearance of low molecular weight nutrients and waste products as well as for rapid systemic exposure of all tissues to circulating erythrocytes, immune cells and hormones. The lymphatic system, on the other hand, provides a unidirectional route for clearance of extravasated interstitial fluid, macromolecules and immune cells from the tissues to the blood circulation via the draining lymph-nodes. Both vascular systems were implicated in providing routes for tumor escape and metastatic dissemination.

Metastatic spread of tumors to sentinel lymph nodes is a critical event in tumor progression, and an important prognostic marker. The lymphatic vessels, though providing a key route for tumor cell dissemination to lymph nodes, are typically absent or collapsed in tumors. However, the rim of primary and metastatic tumors is served by draining lymphatics, which show functional activation and structural expansion in response to tumor derived expression of lymphangiogenic growth factors.

The transcriptional coactivator LEDGF/p75 (also named “PSIP2 isoform”) is induced by environmental stresses. Expression of LEDGF/p75 results in induced expression of LEDGF/p75 target genes (AOP2, αB-crystallin and HSP-27). Consensus LEDGF/p75 binding sites, known as stress response elements (STRE), have been described. LEDGF/p75 is expressed in primary and metastatic cancer cells (Daugaard et al, 2007).

VEGF-C is a protein whose expression is regulated by environmental stresses. VEGF-C has been implicated in peritumor lymphatic remodeling and lymph node metastatic spread for multiple types of cancers (Manila et al., 2002; Skobe et al., 2001). VEGF-C was also shown to be the key lymphangiogenic factor in development and wound healing, mediating its effect through activation of VEGFR-3 on lymphatic endothelial cells. VEGF-C is expressed as a 61 kDa protein, which is subsequently processed by proteolytic enzymes to yield the mature growth factor with increasing affinity to VEGFR-3 on lymphatic endothelial cells.

Prolonged maintenance of tissue homeostasis should include, beyond the acute functional lymphatic response to edema, also a mechanism that would augment lymphatic bed capacity through lymphangiogenesis in response to a chronic increased need for lymphatic clearance. Indeed, VEGF-C expression was shown to be induced by interstitial convection and edema in several experimental models (Boardman and Swartz, 2003; Goldman et al., 2007a; Rutkowski et al., 2006) and in response to inflammation (Baluk et al., 2005).

U.S. Pat. No. 6,750,052 discloses nucleic acids encoding a LEDGF protein, including fragments and biologically functional variants thereof U.S. Pat. No. 6,750,052 further discloses methods for decreasing LEDGF mediated activity in a subject with a cancer that expresses LEDGF. Though U.S. Pat. No. 6,750,052 teaches LEDGF antisesne nucleic acids as preferred agents in decreasing LEDGF activity, there is no indication of specific antisense sequences cable of doing so.

US Patent Application Publication No. 2007/0243191 discloses methods of cancer therapy or diagnosis involving targeting hepatoma-derived growth factor (HDGF) or any HDGF family member, including LEDGF, by antibodies or siRNA that recognize HDGF.

International Patent Application Publication No. WO 2004/029246 discloses an integrase interacting protein, found to be identical to LEDGF, which promotes strand transfer activity of viral integrase, particularly HIV integrase, and uses thereof. Though WO 2004/029246 also relates to inhibition of said integrase interacting protein through antisense and RNA interference among other inhibitors, the use of said inhibitors is targeted for HIV prevention or inhibition.

There remains an unmet medical need for compounds and methods of preventing metastasis of solid tumors. In addition, there remains an unmet medical need for methods of increasing lymphangiogenesis; in particular in conditions of inflammation and edema which require lymphangiogenesis.

### SUMMARY OF THE INVENTION

The present invention provides antisense RNA polynucleotides or oligonucteotides for inhibiting expression of LEDGF/p75, pharmaceutical compositions comprising same and uses thereof for inhibiting tumor growth and/or tumor metastasis. The present invention further provides methods for treating endothelial-cell related conditions or disorders which require lymphatic clearance or lymphangiogenesis, the methods comprise administering to a subject in need of such treatment a pharmaceutical composition comprising a human LEDGF/p75 polypeptide or a functionally active analog or fragment thereof, a nucleic acid encoding same, a vector comprising the nucleic acid or a host cell comprising the vector.

It is now disclosed for the first time that exposure of human lung cancer cells to environmental stress conditions, such as oxidative or thermal stress conditions, resulted in an increased expression of LEDGF/p75 that was associated with an increased expression of VEGF-C.

It is further disclosed that the correlation between VEGF-C mRNA level and LEDFG/p75 mRNA level in ovarian and lung cancer cells was due to the fact that LEDGDF/p75 selectively transactivated VEGF-C gene transcription upon binding to specific sites on VEGF-C promoter.

The present invention discloses that unexpectedly a human natural antisense RNA targeted to human LEDGF/p75 mRNA was able to reduce VEGF-C mRNA and protein levels in human lung cancer cells exposed to oxidative or thermal stress conditions. Moreover, the human antisense RNA was also able to reduce VEGF-C promoter activity in human lung cancer cells when the cells were co-transfected with a VEGF-C promoter, and then exposed to oxidative or thermal stress conditions. The human antisense RNA targeted to human LEDGF/p75 mRNA was found to be functionally active, capable of silencing the expression of VEGF-C.

The present invention further discloses that inoculation in nude mice of human lung carcinoma cells over-expressing LEDGF/p75 induced tumor progression which was associated with an increased expression of VEGF-C as well as with lymphangiogenesis within and around the tumors.

The present invention discloses that surprisingly incubation of ovarian carcinoma cells with the gonadotropins LH or FSH increased mRNA levels of both LEDGF/p75 and VEGF-C. Similar effect on VEGF-C expression was observed in vivo when ovarian carcinoma cells were injected to ovariectomized nude mice.

Thus, the present invention provides specific antisense RNA polynucleotides targeted to a LEDGF/p75 mRNA for inhibiting expression of LEDGF/p75, particularly of human LEDGF/p75, which are useful for inhibiting VEGF-C expression and activity. The antisense RNA polynucleotides consist of up to 700 nucleotides and are highly advantageous for treating diseases associated with increased lymphangiogenesis, particularly for inhibiting tumor growth and/or tumor metastasis.

By virtue of the induction of VEGF-C expression by LEDGF/p75 polypeptide, the present invention further provides methods for treating endothelial-cell related conditions or disorders which require higher capacity of lymphatic clearance and/or lymphangiogenesis, the methods comprise administering to a subject in need of such treatment a pharmaceutical composition comprising as an active agent a LEDGF/p75 polypeptide or a functionally active analog or fragment thereof, an isolated nucleic acid encoding same, a vector comprising the nucleic acid or a host cell comprising the vector, thereby increasing VEGF-C expression and activity which lead to improved lymphatic clearance and/or lymphangiogenesis.

According to a first aspect, the present invention provides an antisense polynucleotide complementary to a LEDGF/p75 mRNA, the antisense polynucleotide comprises the nucleotide sequence as set forth in SEQ ID NO:1 or an active homolog or fragment thereof, said antisense polynucleotide inhibits expression of a LEDGF/p75. It is to be appreciated that the antisense polynucleotide is capable of inhibiting LEDGF/p75 expression and hence VEGF-C expression.

According to one embodiment, the antisense polynucleotide comprises from about 12 to about 700 nucleotides. According to another embodiment, the antisense polynucleotide comprises from about 20 to about 600 nucleotides. Alternatively, the antisense consists of from about 50 to about 300 nucleotides.

According to some embodiments, the antisense polynucleotide is complementary to a human LEDGF/p75 mRNA, said antisense polynucleotide comprises the nucleotide sequence as set forth in any one of SEQ ID NO:2 and SEQ ID NO:3 or an active analog or fragment thereof. According to a certain embodiment, the antisense polynucleotide is complementary to a human LEDGF/p75, said antisense polynucleotide consists of the nucleotide sequence as set forth in SEQ ID NO:1.

According to additional embodiments, the antisense polynucleotide is complementary to a non-human LEDGF/p75 mRNA, said antisense polynucleotide comprises the nucleotide sequence as set forth in any one of SEQ ID NO:4 to SEQ ID NO:6 or an active analog or fragment thereof.

According to another aspect, the present invention provides a pharmaceutical composition comprising as an active agent an antisense polynucleotide complementary to a LEDGF/p75 mRNA, wherein the antisense polynucleotide comprises the nucleotide sequence as set forth in SEQ ID NO:1 or an active homolog or fragment thereof; further comprising a pharmaceutically acceptable carrier.

According to some embodiments, the antisense polynucleotide within the pharmaceutical composition comprises from about 12 to about 700 nucleotides. According to another embodiment, the antisense polynucleotide within the pharmaceutical composition comprises from about 20 to about 600 nucleotides. Alternatively, the antisense polynucleotide consists from about 50 to about 300 nucleotides.

According to additional embodiments, the antisense polynucleotide within the pharmaceutical composition is complementary to a human LEDGF/p75 mRNA, said antisense polynucleotide comprises the nucleotide sequence as set forth in any one of SEQ ID NO:2 and SEQ ID NO:3, or an active homolog or fragment thereof. According to a certain embodiment, the antisense polynucleotide complementary to a human LEDGF/p75 within the pharmaceutical composition consists of the nucleotide sequence as set forth in SEQ ID NO: 1. According to further embodiments, the antisense polynucleotide within the pharmaceutical composition is complementary to a non human LEDGF/p75, said antisense polynucleotide comprises the nucleotide sequence as set forth in any one SEQ ID NO:4 to SEQ ID NO:6 or an active analog or fragment thereof.

According to some embodiments, the pharmaceutical composition is formulated in a form selected from the group consisting of s a solution, suspension, emulsion, powder, cream, lotion, gel, foam, spray, or aerosol.

According to a further aspect, the present invention provides a method for inhibiting tumor progression or tumor metastasis in a subject comprising administering to the subject in need of such treatment a pharmaceutical composition comprising a therapeutically effective amount of an antisense polynucleotide complementary to a LEDGF/p75 mRNA, the antisense polynucleotide comprises the nucleotide sequence as set forth in SEQ ID NO:1 or an active homolog or fragment thereof; further comprising a pharmaceutically acceptable carrier.

According to some embodiments, the antisense polynucleotide useful for inhibiting tumor progression or tumor metastasis comprises from about 12 to about 700 nucleotides, or from about 20 to about 600 nucleotides. Alternatively, the antisense polynucleotide consists of from about 50 to about 300 nucleotides.

According to additional embodiments, the antisense polynucleotide useful for inhibiting tumor progression or tumor metastasis comprises the nucleotide sequence as set forth in SEQ ID NO:2 to SEQ ID NO:6 or an active homolog or fragment thereof. According to a certain embodiment, the antisense polynucleotide useful for inhibiting tumor progression or tumor metastasis consists of the nucleotide sequence as set forth in SEQ ID NO:1.

According to some embodiments, the tumor to be treated is solid tumor selected from the group consisting of ovarian carcinoma, lung carcinoma including small cell lung carcinoma, non-small and large cell lung carcinoma, breast carcinoma, prostate carcinoma, pancreas carcinoma, liver carcinoma, colon carcinoma, hepatocellular carcinoma, bladder carcinoma, rectal carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, glioma, astrocyoma, medulloblastoma, craniopharyngioma, ependymoma, pinealoma, retinoblastoma, and neuroblastoma.

According to certain embodiments, the tumor to be treated is lung carcinoma or ovarian carcinoma.

According to additional embodiments, the tumor to be treated is non-solid tumor selected from the group consisting of leukemia and lymphoma. According to further embodiments, the non-solid tumor is selected from the group consisting of acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

According to further embodiments, the route of administering the pharmaceutical composition is selected from the group consisting of intravenous, subcutaneous, intramuscular, intraperitoneal, oral, nasal, rectal, vaginal, and topical. According to a certain embodiment, the route of administering the pharmaceutical composition is by injection into the tumor or adjacent to the tumor.

According to further aspect, the present invention provides a method for reducing or inhibiting VEGF-C expression in a cell comprising contacting the cell with an antisense polynucleotide according to the principles of the present invention, thereby reducing or inhibiting VEGF-C expression.

According to another aspect, the present invention provides use of an antisense polynucleotide according to the principles of the present invention, for the preparation of a medicament for inhibiting tumor progression or tumor metastasis.

According to yet further aspect, the present invention provides a method for treating an endothelial cell related condition or disorder in a subject comprising administering to a subject in need of such treatment a pharmaceutical composition comprising a therapeutically effective amount of an active agent selected from the group consisting of: (a) an isolated LEDGF/p75 polypeptide comprising the amino acid sequence as set forth in SEQ ID NO:20 or an active analog or fragment thereof; (b) an isolated nucleic acid molecule encoding LEDGF/p75 polypeptide, the LEDGF/p75 comprises the amino acid sequence as set forth in SEQ ID NO:20 or an active analog or fragment thereof; (c) an expression vector comprising the isolated nucleic acid molecule of (b); and (d) a host cell transfected with the expression vector of (c); further comprising a pharmaceutically acceptable carrier. Preferably, the subject is a human. It is to be understood that the method of treatment of the present invention is applicable for treating endothelial cell related conditions or disorders which require improved lymphatic clearance and/or lymphangiognesis and/or angiogenesis.

According to some embodiments, the isolated LEDGF/p75 polypeptide useful for treating an endothelial cell related condition or disorder comprises the amino acid sequence as set forth in SEQ ID NO:21 or an active analog or fragment thereof. According to a certain embodiment, the isolated LEDGF/p75 polypeptide useful for treating endothelial cell related condition or disorder consists of the amino acid as set forth in SEQ ID NO: 20.

According to additional embodiments, the isolated nucleic acid molecule encoding LEDGF/p75 comprises the nucleotide sequence selected from the group consisting of SEQ ID NO:22 to SEQ ID NO:24. According to a certain embodiment, the isolated nucleic acid molecule encoding LEDGF/p75 consists of the nucleotide sequence as set forth in SEQ ID NO:22.

According to further embodiments, the condition to be treated is an inflammation. According to yet further embodiments, the inflammation is associated with an inflammatory disease. According to still further embodiments, the inflammation is associated with an autoimmune disease. According to still further embodiments, the inflammation is associated with an injury. According to yet further embodiments, the inflammation is associated with an infectious disease. According to still further embodiments, the inflammation is associated with transplantation of a graft. According to yet further embodiments, the inflammation is associated with a degenerative neurological disease.

According to additional embodiment, the condition is edema. According to some embodiments, the edema is lymphedema or tissue edema.

According to another aspect, the present invention provides use of a compound selected from the group consisting of: (a) an isolated LEDGF/p75 polypeptide comprising the amino acid sequence as set forth in SEQ ID NO:20 or an active analog or fragment thereof; (b) an isolated nucleic acid molecule encoding LEDGF/p75 polypeptide, the LEDGF/p75 polypeptide comprises the amino acid sequence as set forth in SEQ ID NO:20 or an active analog or fragment thereof; (c) an expression vector comprising the isolated nucleic acid molecule of (b); and (d) a host cell transfected with the expression vector of (c); for the preparation of a medicament for treating an endothelial cell related condition or disorder according to the principles of the present invention.

According to some embodiments, the pharmaceutical composition is formulated in a form selected from the group consisting of a solution, suspension, emulsion, powder, cream, lotion, gel, foam, spray, and aerosol.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1. Co-Expression of VEGF-C and LEDGF in cancer cells. (A) VEGF-C, LEDGF and VEGF-A mRNA expression was analyzed by RT-PCR in H1299 and A594 lung cancer cells. VEGF-C, LEDGF and VEGF-A relative mRNA expression normalized against GAPDH (B-D respectively). (E) VEGF-C, LEDGF and VEGF-A mRNA expression was analyzed by RT-PCR in MLS, ES2 and SKOV-3 human ovarian cancer cell lines. VEGF-C, LEDGF and VEGF-A relative mRNA expression normalized against GAPDH (F-H respectively).

FIG. 2. Over-expression of LEDGF/p75 triggers transcription of VEGF-C mRNA. (A) H1299 cells, stably transfected either with vector expressing the luciferase gene alone (pIRES-Luc) or with a construct encoding for both luciferase and rat LEDGF tagged with an influenza virus hemagglutinin epitope (pIRES-Luc-LEDGF). Immunoblot assay of LEDGF expression levels of puromycin-resistant stably transfected pools (lanes 1-2) and individual clones (clone 3-7) using anti-HA antibodies. (B) RT-PCR analysis of expression of human VEGF-C, VEGF-A, GAPDH and transfected rat LEDGF genes was carried out on total RNA extracted from the above stable transfected pools and individual clones. VEGF-C, LEDGF and VEGF-A relative mRNA expression normalized against GAPDH(C-E respectively).

FIG. 3. Identification of LEDGF/p75 binding sites in the VEGF-C Gene (A) Candidate binding sites (underlined) were identified in a of 468 by 5′-flanking region of human VEGF-C gene. Nucleotide sequences are numbered in relation to the ATG, which is designated “+1.” (B) Conservation among species of a putative binding unit (−426 to −407; human chr4:177,950,885-177,950,866) for LEDGF was identified using the conservation track of the UCSC genome browser, and the positions of mismatches are indicated.

FIG. 4. A luciferase reporter gene containing the 5′-flanking region of human VEGF-C gene of FIG. 3A (pVEGF-C-Luc) was transiently cotransfected together with control empty vector (pIRES) or with a construct encoding LEDGF/p75 into rat glioma C6 or COS7 cells (A and B respectively). Luciferase activity under each experimental condition was scaled relative to the activity in control cells (average±SD, n=3). (C and D, upper panel) Western blot analysis carried out using anti-HA antibodies of protein extracted from C6 rat glioma (C) or H1299 (D) cells stably expressing rat LEDGF protein tagged with hemagglutinin (HA) epitope (pIRES-LEDGF) following selection with puromycin. (C and D lower panel). C6 (C) or H2299 (D) cells transfected with pIRES-LEDGF or empty control vector were transiently transfected with pVEGF-C-Luc.

FIG. 5. Importance of the conserved STREs for VEGF-C promoter activity. (A) Wild type (wt) and two mutated (m1 and m2) sequences of the conserved LEDGF/p75 binding sites of FIG. 3B are presented, and the G to A substitutions are indicated in gray. (B) H1299 cells were transiently cotransfected with pVEGF-Cwt-Luc, pVEGF-Cm1-Luc, or pVEGF-Cm2-Luc together with LEDGF/p75 expression vector or empty vector. Average±SD (n=3) is depicted. (C) ChIP assays using specific primers for 468 by VEGF-C promoter (FIG. 3A) and non-specific (ns) or anti-LEDGF/p75 antibodies.

FIG. 6. VEGF-C expression is induced by oxidative stress. (A) VEGF-C and LEDGF-p75 mRNA expression analyzed by RT-PCR in H1299 cells exposed for 1 and 6 hours to 0.2 mM H₂0₂ relative to untreated cells. (B) Relative intensity of mRNA expression, normalized against GAPDH. (C) Immunoblot assay of VEGF-C, LEDGF and ?-tubulin in H1299 cells treated with 0.2 mM H₂0₂ for 6 or 12 hours or untreated. (D) Luciferase reporter assay utilized a construct containing a wild-type 468 by VEGF-C gene fragment (pVEGF-Cwt-Luc) or mutated constructs (pVEGF-Cm1-Luc and pVEGF-Cm2-Luc). H1299 cells were stimulated 24 hr post-transfection with 0.2 mM H₂0₂ or untreated (average±SD, n=3). (E) Chromatin from cells exposed to 0.2 mM H₂O₂ for 1 and 4 h or untreated (0 h) was immunoprecipitated (ChIP) with anti LEDGF/p75 specific antibody and analyzed by PCR using primers spanning the 468 by VEGF-C promoter (FIG. 3A).

FIG. 7. VEGF-C expression is induced by thermal stress. (A) VEGF-C and LEDGF/p75 mRNA expression was analyzed by RT-PCR in H1299 cells either incubated at 42° C. for 6 hours, followed by a 6-hr incubation at 37° C. (12 hr) or left at 37° C. (0 hr). (B) Relative intensity of mRNA expression, normalized to GAPDH. (C) Immunoblot assay of VEGF-C, LEDGF and ?-tubulin in H1299 cells treated as indicated in (A). (D) Luciferase reporter assay carried out with pVEGF-Cwt-Luc, pVEGF-Cm1-Luc, and pVEGF-Cm2-Luc. H1299 cells were heat activated (42° C.) for 6 hr and then maintained for 6 hr at 37° C. Average±SD (n=3) is depicted. (E) Chromatin from cells heat activated (42° C.) for the indicated time was immunoprecipitated (ChIP) with anti LEDGF/p75 specific antibody and analyzed by PCR using primers spanning the 468 by VEGF-C promoter (FIG. 3A).

FIG. 8. Existence of LEDGF/p75 cis-native antisense transcripts (cis-NAT). (A) Illustration of genomic structure of LEDGF/p52 (AF339083) and LEDGF/p75 (NM_{—}133948.4) variants. Black boxes indicate exons and the connecting line introns. (B) Diagram of the mouse (cis-NAT). Accession numbers (in order of start position from left to right): AK140469, AK038357, AK020824, AK171985, AK053153, AK042735, AK143096. (C) Expansion of the genomic region covered by the (cis-NAT) AK042735. The 214 bp-long exon probe designed against the cis-NAT of LEDGF is indicated by an empty box. Accession numbers of equivalent transcripts in other species: Rat-CB576984; Human-AV716383, DA171947; Cow-CK778664, BF654277. (D) RT-PCR analysis of RNA from mouse, rat and human origin performed with LEDGF/p75 antisense specific primers designed to encompass the exon probe (indicated by open box in (A, C) and an arrow in (A).

FIG. 9. A cis-natural antisense RNA of LEDGF is functionally active. (A) Analysis of VEGF-C and LEDGF sense and antisense transcripts by specific RT-PCR in control A549 (−) or stably over-expressing LEDGF antisense construct (+). For amplification of antisense transcripts, sense-specific primers were added to reverse transcription, whereas for the detection of the sense transcripts, antisense primers were added. Relative intensity of VEGF-C, LEDGF sense and LEDGF antisense mRNA expression normalized against GAPDH (B-D respectively). (E) Immunoblot assay of VEGF-C, LEDGF/p75 and ?-tubulin protein in A549 cells stably transfected with empty vector (−) or a construct expressing LEDGF antisense (+). (F-G) H1299 cells were co-transfected with VEGF-C-luciferase reporter and empty vector (pIRES) or vector expressing LEDGF antisense (pIRES-LEDGFas). Cells were incubated with 0.2 mM H₂0₂ for 12 hr (F) or heated to 42° C. for 6 h, followed by 6 h at 37° C. (G) (average±SD, n=3).

FIG. 10. VEGF-C expression is induced in H1299 tumors over-expressing LEDGF. (A-B) In vivo bioluminescence imaging ten days following inoculation of CD-1 nude mice (5×10⁶ cells/mouse) with H1299-Luc control cells (A) or H1299-Luc over-expressing LEDGF (B, see FIG. 2) (C) Subcutaneous tumors three weeks post-inoculation. (D) Table summarizing the in vivo study. (E) Neutral red (NR) cytotoxicity assay carried out in vitro with H1299-Luc and H1299-Luc-LEDGF cells maintained up to 72 hours in the presence of 1% or 10% fetal bovine serum (FBS) to evaluate proliferation rate (average±SD, n=4). (F-Q) Ex-vivo analysis of subcutaneous tumors excised from the mice inoculated either with control H1299-Luc (F, H, J, L, N, P) or H1299-Luc-LEDGF (G, I, K, M, O, Q) cells. (F-G) Histologic sections stained with hematoxylin-eosin. (H-I) ISH analysis using VEGF-C as probe. (J-K) Expression of HA-LEDGF in H1299-Luc-LEDGF tumors, visualized by staining of histologic sections with anti-HA antibody. (L-M) Immunohistochemical (IHC) staining of lymphatic endothelial cells using anti-LYVE1 antibodies. (N-O) IHC staining of vascular smooth muscle cells and pericytes with anti-alpha smooth muscle actin (alpha-SMA) antibodies. (P-Q) TdT-mediated dUTP-biotin nick end labeling (TUNEL) showed no difference in the apoptotic ratio between control and LEDGF-overexpressing tumors. Scalebar=50 ?m.

FIG. 11. Overexpression of LEDGF augments migration and invasion rate of H1299 cells in vitro. (A) Cell migration assay with 111299 encoding LEDGF and luciferase (H1299-Luc-LEDGF) and control cells transfected with H1299-Luc. (B) Cells that had migrated through the filter were stained, and cell density was determined by image analysis (using ImageJ). (C) Invasion of H1299-Luc-LEDGF and 111299-Luc through Matrigel™. Cells were plated in transwell invasion chambers coated with Matrigel, and 12 hours later, cells that had migrated through the filter were stained and counted using ImageJ (D). (Three independent experiments each carried out with at least triplicates; star indicates p<0.01).

FIG. 12. LH stimulation induces VEGF-C, LEDGF and LEDGF antisense RNA expression in vitro. ES2 cells stimulated with 1 ng/ml LH for the indicated times. VEGF-C, LEDGF and LEDGF antisense RNA expression was analyzed by RT-PCR and normalized against GAPDH (A-C respectively).

FIG. 13. FSH stimulation induces VEGF-C, LEDGF and LEDGF antisense RNA expression in vitro. ES2 cells stimulated with 1 ng/ml FSH for the indicated times. VEGF-C, LEDGF and LEDGF antisense RNA expression was analyzed by RT-PCR and normalized against GAPDH (A-C respectively).

FIG. 14. In vitro hormonal stimulation induces VEGF-C activation, probably in a LEDGF/p75 dependent manner. (A) Western blot analysis carried out on ES2 cells stimulated with 1 ng/ml LH or 1 ng/ml FSH. (B) VEGF-C promoter activity examined by a luciferase assay. (C) LEDGF binding to VEGF-C promoter was analyzed by ChIP assay.

FIG. 15. Hormonal stimulation induces VEGF-promoter activation in vivo. (A). Ovariectomized and control mice were subcutaneously injected with ES2 cells stably transfected with a construct containing the luciferase gene under the regulation of the VEGFC promoter. The VEGF-C promoter activity was measured in vivo using the IVIS system. (B) The total flux of photons from the tumor area was measured in the two groups of mice. (C) Tumor dimensions were measured manually and the tumor volume was calculated.

### DETAILED DESCRIPTION OF THE INVENTION

The present invention provides compounds and methods for modulating the expression of Lens Epithelial Cell Derived Growth Factor (LEDGF/p75) in target cells. Particularly, the present invention provides antisense RNA polynucleotides targeted to LEDGF/p75 mRNA which are capable of reducing LEDGF/p75 expression, thereby reducing VEGF-C mRNA and polypeptide levels. As VEGF-C is known to be a potent lymphangiogenic factor, the antisense polynucleotides are useful for inhibiting tumor progression and tumor metastasis.

The present invention provides an antisense polynucleotide targeted to human LEDGF/p75 mRNA comprising the nucleotide sequence as set forth in SEQ ID NO:1 as follows:

GACCCTGTTTGTTCCTTCTCTAGCTTTTTGTTTGGCCCTTTCTTCCCT
TGATCTTTGGTTTTATTCGCTTCCTCATGCTGTCTTTGTTCAGCAAGA
GATTTATTCAGCACTTGGGTGATCACGGAATCTCCTTCACCAACCAAG
AACATGTTCTTAAACTTGTTATACAACATTGTAGACTTTTCCATGATT
ACCTGACTAACTTTGAATCGCCGTAT;

or an active homolog or fragment thereof.

The present invention further provides additional exemplary non-limiting LEDGF/p75 antisense polynucleotides comprising the nucleotide sequence as disclosed in table 1, or active homologs or fragments thereof

TABLE 1

| SEQ ID NO: | Species | Accession No. |
|---|---|---|
| 2 | Human | DA171947 |
| 3 | Human | AV716383 |
| 4 | Rat | CB576984 |
| 5 | Cow | CK778664 |
| 6 | Cow | BF654277 |
| 7 | Mouse | AK042735 |

It is to be explicitly appreciated that known antisense oligonucleotides or polynucleotides are excluded from the novel compounds of the present invention, but are disclosed and claimed for the novel uses as disclosed herein.

The antisense oligonucleotides or polynucleotides of the invention specifically hybridize with one or more nucleic acid molecules encoding LEDGF/p75 polypeptide. As used herein, the term “nucleic acid molecule encoding LEDGF/p75 polypeptide” has been used for convenience to encompass DNA encoding LEDGF/p75, RNA (including pre-mRNA and mRNA or portions thereof) transcribed from such DNA, and also cDNA derived from such RNA. The hybridization of an antisense with its target nucleic acid is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable.

The functions of DNA that can be inhibited or interfered with an antisense oligonucleotide or polynucleotide can include replication and transcription. The functions of RNA to be interfered with an antisense oligonucleotide or polynucleotide can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In the present invention the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances. Stringent conditions under which oligomeric compounds hybridize to a target sequence are determined by the nature and composition of the oligomeric compounds and the assays in which they are being investigated.

“Complementary” as used herein, refers to the capacity for precise pairing between one nucleobase of an oligomeric compound to its target nucleic acid. For example, if a nucleobase at a certain position of an oligonucleotide or polynucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

The potency of antisense oligonucleotides for inhibiting LEDGF/p75 expression can be enhanced using various methods including addition of polylysine, encapsulation into liposomes (antibody targeted, cationic acid, Sendai virus derived, etc.) or into nanoparticles in order to deliver the oligonucleotides into cells. Other techniques for enhancing the antisense capacity of oligonucleotides exist, such as the conjugation of the antisense oligonucleotides for example to “cell penetrating peptides” (Manoharan, M. Antisense Nucleic Acid Drug Dev. 2002:12(2): 103-128/Juliano. R. L. Curr. Opin. Mol. Ther. 2000: 2(3): 297-303).

It is appreciated that the present invention further encompasses short polynucleotide antisense sequences comprising from about 8 to 30 nucleotides, alternatively from about 10 to 28 nucleotides, further alternatively from about 12 to 26 nucleotides.

The present invention further provides methods for inhibiting VEGF-C expression through the use of a LEDGF/p75 RNAi molecule. “LEDGF/p75 RNAi nucleotide” as used herein, refers to an RNAi molecule with activity against LEDGF/p75 expression. The LEDGF/p75 RNAi of the present invention may be any RNAi capable of reducing expression of LEDGF/p75 in a target cell, and can be e.g. an siRNA, rasiRNA, shRNA, or miRNA.

It is understood in the art that the LEDGF/p75 RNAi molecule may be a fragment or a fragment of a homolog of the antisense sequences of the present invention (SEQ ID NO:1 to SEQ ID NO:7).

Small interfering RNAS (siRNAs) are 21-25 by double strand RNA (dsRNA) with dinucleotide 3′ overhangs that are formed in the cell from longer dsRNA molecules. The fully assembled RNA-induced silencing complex (RISC) contains only one strand of the siRNA, the guide strand. The guide strand is thought to provide target specificity for RISC-mediated cleavage through perfect base pairing with the mRNA target. Endogenous siRNAs have been identified in plants, fungi, and animals. These siRNAs are derived in vivo from perfectly base-paired dsRNA precursors comprised of two distinct RNA strands. In many cases, endogenous siRNAs originate from repetitive elements within the genome, such as heterochromatic regions at centromeres and telomeres, and are therefore known as repeat-associated siRNAs (rasiRNAs).

Anti-LEDGF/p75 siRNA are well known in the art, and are described, for example, in Vandekerckhove L et al. (Transient and stable knockdown of the integrase cofactor LEDGF/p75 reveals its role in the replication cycle of human immunodeficiency virus. J Virol 80(4):1886-96, 2006).

Representative, non-limiting examples of anti-LEDGF/p75 siRNA are:

guuccugauggagcuguaatt (sense) +
uuacagcuccaucaggaactt (antisense);
SEQ ID NO: 8 and 9, respectively).
cagcccuguccuucagagatt (sense) +
ttgucgggacaggaagucucu (antisense);
SEQ ID NO: 10 and 11, respectively).
agacagcaugaggaagcgatt (sense) +
ttucugucguacuccuucgcu (antisense);
SEQ ID NO: 12 and 13, respectively).

In another embodiment, an anti-LEDGF/75 siRNA is created from the oligonucleotides ggacatgatgaccttgttgaaagctttcaacaaggtcatcatgtccctttttg (SEQ ID NO: 14) and aattcaaaaagggacatgatgaccttgttgaaagattcaacaaggtcatcatgtcc (SEQ ID NO: 15), as described in Devroe, E., and P. A. Silver. 2002. Retrovirus-delivered siRNA. BMC Biotechnol 2:15.

In another embodiment, an anti-LEDGF/75 siRNA is created from the oligonucleotides gatcccagacagcatgaggaagcgattcaagagatcgcttcctcatgctgtctttttttggaaa (SEQ ID NO: 16) and agatttccaaaaaaagacagcatgaggaagcgatctcttgaatcgcttcctcatgctgtctgg (SEQ ID NO: 17) or from the oligonucleotides gatcccgactctaaatggaggtttcaagagaagatcctccatttagagtcttttttggaa (SEQ ID NO: 18) and agatttccaaaaaagactctaaatggaggatcttctcttgaaagatcctccatttagagtcg (SEQ ID NO: 19) as described in Llano M et al (LEDGF/p75 determines cellular trafficking of diverse lentiviral but not murine oncoretroviral integrase proteins and is a component of functional lentiviral preintegration complexes. J Virol 78(17):9524-37, 2004).

Thus, the present invention provides a method for reducing or inhibiting VEGF-C expression in a cell comprising contacting the cell with a siRNA oligonucleotide comprising a sense RNA strand and an antisense RNA strand, wherein the sense and the antisense RNA strands form an RNA duplex, and wherein the sense strand comprises the nucleotide sequence as set forth in any one of SEQ ID NOs:8, 10, 12, 14, 16, and 18, and wherein the antisense strand comprises the nucleotide sequence as set forth in any one of SEQ ID NOs:9, 11, 13, 15, 17, and 19, respectively.

MicroRNA (miRNAs) are 19-23 nt single-stranded RNAs, originating from single-stranded precursor transcripts that are characterized by imperfectly base-paired hairpins. miRNAs function in a silencing complex that is similar, if not identical, to RISC.

Short hairpins RNA (shRNAs) are used in plasmid- or vector-based approaches for supplying siRNAs to cells to produce stable gene silencing. A strong promoter is often used to drive transcription of a target sequence designed to form hairpins and loops of variable length, which are then processed to siRNAs by the cellular RNAi machinery.

The present invention further provides uses of a LEDGF/p75 polypeptide, an isolated nucleic acid encoding same, expression vector comprising the nucleic acid, and a host cell transfected with the expression vector for treating endothelial cell related conditions or disorders.

The amino acid sequence of human LEDGF/p75 polypeptide is presented as SEQ ID NO:20. The nucleotide sequence of human LEDGF/p75 cDNA is presented as SEQ ID NO:22 and SEQ ID NO:23.

The LEDGF/p75 polypeptide or nucleic acid useful for the methods of the present invention can be any LEDGF/p75 polypeptide or any nucleic acid encoding same. Exemplary, non-limiting LEDGF/p75 sequences are disclosed herein in Table 2.

TABLE 2

| SEQ ID NO: | Species | Sequence | Accession No. |
|---|---|---|---|
| 20 | Human | Polypeptide | AF063020 |
| 21 | Mouse | Polypeptide | NM_133948.4 |
| 22 | Human | Nucleic acid | AF063020 |
| 23 | Human | Nucleic acid | NM_033222.3 |
| 24 | Mouse | Nucleic acid | NM_133948.4 |

The present invention provides uses of an isolated LEDGF/p75 polypeptide, or an analog or fragment thereof.

The term “polypeptide” as used herein refers to a linear series of natural, non-natural and/or chemically modified amino acid residues connected one to the other by peptide bonds. The amino acid residues are represented throughout the specification and claims by either one or three-letter codes, as is commonly known in the art.

The term “analog” refers to a polypeptide comprising at least one altered amino acid residue by an amino acid substitution, addition, deletion, or chemical modification, as compared with the native polypeptide. Polypeptide analogs include amino acid substitutions and/or additions with naturally occurring amino acid residues, and chemical modifications such as, for example, enzymatic modifications, typically present in nature. Polypeptide analogs also include amino acid substitutions and/or additions with non-natural amino acid residues, and chemical modifications which do not occur in nature.

In general, analogs typically will share at least 50% amino acid identity to the native sequences disclosed in the present invention, in some instances the analogs will share at least 60% amino acid identity, at least 70%, 80%, 90%, and in still other instances the analogs will share at least 95% amino acid identity to the native polypeptides.

The term “fragment” as used herein refers to a portion of a polypeptide, or polypeptide analog which retains the biological activity of the native polypeptide, i.e., induction of lymphangiogenesis and/or improvement in lymphatic clearance so as to treat endothelial cell related conditions or disorders.

For brevity, the term “polypeptide” used in the specification and claims includes analogs and fragments of the polypeptides. It is to be appreciated that the analogs and fragments should be active, i.e., their biological activity should be similar to or even higher than that of the native polypeptide, such as, for example, their capability to induce lymphangiogenesis and/or angiogenesis and/or to increase lymphatic clearance.

By using “amino acid substitutions”, it is meant that functionally equivalent amino acid residues are substituted for residues within the sequence resulting in a silent change. The term “functionally equivalent” means, for example, a group of amino acids having similar polarity, similar charge, or similar hydrophobicity. For example, one or more amino acid residues within the sequence can be substituted by another amino acid of a similar polarity, which acts as a functional equivalent, resulting in a silent alteration. Substitutes for an amino acid within the sequence can be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions. Additionally, a non-conservative substitution can be made in an amino acid that does not contribute to the biological activity of the polypeptide. Such non-conservative substitutions are also encompassed within the term “amino acid substitution”, as used herein. It will be appreciated that the present invention further encompasses polypeptides in which at least one amino acid is substituted by another amino acid to produce polypeptide analogs having increased stability or higher half life as compared to the native polypeptides.

The present invention encompasses polypeptide hydrates. The term “hydrate” includes, but is not limited to, hemihydrate, monohydrate, dihydrate, trihydrate, and the like.

The polypeptides of the present invention can be produced by various methods known in the art, including recombinant production or synthetic production. Recombinant production can be achieved by the use of an isolated polynucleotide encoding the polypeptide of the present invention, or a fragment, or analog thereof, the isolated polynucleotide operably linked to a promoter for the expression of the polynucleotide. Optionally, a signal peptide and a regulator of the promoter are added. The construct comprising the polynucleotide encoding the polypeptides of the present invention, or a fragment, or analog thereof, the promoter, and optionally the regulator can be placed in a vector, such as a plasmid, virus or phage vector. The vector can be used to transfect or transform a host cell, e.g., a bacterial, yeast, insect, or mammalian cell. The vector can also be introduced into a transgenic animal such as, for example, a transgenic mouse.

Alternatively, the polypeptide can be produced synthetically. Synthetic production of peptides is well known in the art (see, for example, Bodanszky, 1984, Principles of Peptide Synthesis, Springer-Verlag, Heidelberg), such as via solid-phase synthesis (see, for example, Merrifield, 1963, J. Am. Chem. Soc. 85:2149-2154, the contents of which are hereby incorporated by reference in their entirety).

The present invention also encompasses polypeptide analogs in which free amino groups have been derivatized to form amine hydrochlorides, p-toluene sulfonylamino groups, carbobenzoxyamino groups, t-butyloxycarbonylamino groups, chloroacetylamino groups or formylamino groups. Free carboxyl groups may be derivatized to form, for example, salts, amides, methyl and ethyl esters or other types of esters or hydrazides. The imidazole nitrogen of histidine can be derivatized to form N-im-benzylhistidine.

Included within the scope of the invention are polypeptide conjugates comprising the polypeptides of the present invention joined at their amino or carboxy-terminus or at one of the side chains via a peptide bond to an amino acid sequence of a different protein. Additionally or alternatively, the polypeptides of the present invention can be joined to another moiety such as, for example, a fatty acid, a sugar moiety, and any known moiety that facilitate membrane or cell penetration. Conjugates comprising polypeptides of the invention and a protein can be made by protein synthesis, e.g., by use of a peptide synthesizer, or by ligating the appropriate nucleic acid sequences encoding the desired amino acid sequences to each other by methods known in the art, in the proper coding frame, and expressing the conjugate by methods commonly known in the art.

The term “nucleic acid” as used herein refers to a polynucleotide of DNA or RNA of genomic or synthetic origin, which may be single- or double-stranded, and represent the sense or antisense strand. The term should also be understood to include, as equivalents, homologs of either RNA or DNA made from nucleotide analogs, and, as applicable to the embodiment being described.

An “isolated nucleic acid” refers to a polynucleotide segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to polynucleotides, which have been substantially purified from other components, which naturally accompany the polynucleotide in the cell, e.g., RNA or DNA or proteins. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence, and RNA such as mRNA.

The term “encoding” refers to the inherent property of specific sequences of nucleotides in an isolated polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a peptide or protein if transcription and translation of mRNA corresponding to that gene produces the peptide or protein in a cell or other biological system. Both the coding strand, the nucleotide sequence of which is identical to the mRNA sequence, and the non-coding strand, used as the template for transcription of a gene or cDNA, can be referred to as encoding the peptide or protein or other product of that gene or cDNA.

The term “homolog” refers to an oligonucleotide or polynucleotide or nucleic acid comprising at least one altered nucleotide base (nucleobase) by a nucleotide base substitution, addition, deletion, or chemical modification, as compared with the native oligonucleotide or polynucleotide or nucleic acid. In general, homologs typically will share at least 50% nucleotide identity to the native sequences of the present invention, in some instances the homologs will share at least 60% nucleotide identity, at least 70%, 80%, 90%, and in still other instances the homologs will share at least 95% nucleotide identity. The homology can be calculated using various, publicly available software tools developed by NCBI (Bethesda, Md.) that can be obtained through the internet. Exemplary tools include the BLAST system. Pairwise and ClustalW alignments (BLOSUM30 matrix setting) as well as Kyte-Doolittle hydropathic analysis can be obtained using the MacVetor sequence analysis software (Oxford Molecular Group). Watson-Crick complements of the foregoing nucleic acids also are embraced by the invention. It is to be appreciated that the homologs of the present invention should exert similar or even higher activity than that exerted by the native or disclosed polynucleotide.

The invention also includes degenerate nucleic acids which include alternative codons isolated from a cDNA library prepared from one or more of the tissues in which LEDGF expression is abundant, using standard colony hybridization techniques.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating LEDGF polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

A nucleic acid of the present invention can be expressed as a secreted polypeptide where the polypeptide of the present invention is isolated from the medium in which the host cell containing the nucleic acid is grown, or the nucleic acid can be expressed as an intracellular polypeptide by deleting the leader or other peptides, in which case the polypeptide of the present invention is isolated from the host cells. The polypeptide of the present invention so isolated is then purified by standard protein purification methods known in the art.

The polypeptides of the present invention, analogs, or fragments thereof can also be provided to the tissue of interest by transferring an expression vector comprising an isolated nucleic acid encoding the polypeptide of the present invention, or an analog, or fragment thereof to cells associated with the tissue of interest. The cells produce the polypeptide such that it is suitably provided to the cells within the tissue to exert a biological activity such as, for example, to modulate VEGF-C expression within the tissue of interest.

An “expression vector” as used herein refers to a nucleic acid molecule capable of replication and expressing a gene of interest when transformed, transfected or transduced into a host cell. The expression vectors comprise one or more phenotypic selectable markers and an origin of replication to ensure maintenance of the vector and to, if desired, provide amplification within the host. Selectable markers include, for example, sequences conferring antibiotic resistance markers, which may be used to obtain successful transformants by selection, such as ampicillin, tetracycline and kanamycin resistance sequences, or supply critical nutrients not available from complex media. Suitable expression vectors may be plasmids derived, for example, from pBR322 or various pUC plasmids, which are commercially available. Other expression vectors may be derived from bacteriophage, phagemid, or cosmid expression vectors, all of which are described in sections 1.12-1.20 of Sambrook et al., (Molecular Cloning: A Laboratory Manual. 3^{rd} edn., 2001, Cold Spring Harbor Laboratory Press). Isolated plasmids and DNA fragments are cleaved, tailored, and ligated together in a specific order to generate the desired vectors, as is well known in the art (see, for example, Sambrook et al., ibid).

A nucleic acid molecule can be produced using recombinant DNA technology (e.g., polymerase chain reaction (PCR) amplification, cloning) or chemical synthesis. Nucleic acid sequences include natural nucleic acid sequences and homologs thereof, comprising, but not limited to, natural allelic variants and modified nucleic acid sequences in which nucleotides have been inserted, deleted, substituted, and/or inverted in such a manner that such modifications do not substantially interfere with the nucleic acid molecule's ability to encode the recombinant polypeptides of the present invention.

The expression vector according to the principles of the present invention further comprises a promoter. In the context of the present invention, the promoter must be able to drive the expression of the peptide within the cells. Many viral promoters are appropriate for use in such an expression vector (e.g., retroviral ITRs, LTRs, immediate early viral promoters (IEp) (such as herpes virus IEp (e.g., ICP4-IEp and ICP0-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters). Other suitable promoters are eukaryotic promoters, which contain enhancer sequences (e.g., the rabbit ?-globin regulatory elements), constitutively active promoters (e.g., the ?-actin promoter, etc.), signal and/or tissue specific promoters (e.g., inducible and/or repressible promoters, such as a promoter responsive to TNF or RU486, the metallothionine promoter, etc.), and tumor-specific promoters.

Within the expression vector, the nucleic acid encoding the polypeptide of the present invention, or an analog, or fragment thereof and the promoter are operably linked such that the promoter is able to drive the expression of the nucleic acid. As long as this operable linkage is maintained, the expression vector can include more than one gene, such as multiple genes separated by internal ribosome entry sites (IRES). Furthermore, the expression vector can optionally include other elements, such as splice sites, polyadenylation sequences, transcriptional regulatory elements (e.g., enhancers, silencers, etc.), or other sequences.

The expression vectors are introduced into the cells in a manner such that they are capable of expressing the isolated nucleic acid encoding the polypeptides of the present invention contained therein. Any suitable vector can be so employed, many of which are known in the art. Examples of such vectors include naked DNA vectors (such as oligonucleotides or plasmids), viral vectors such as adeno-associated viral vectors (Berns et al., 1995, Ann. N.Y. Acad. Sci. 772:95-104, the contents of which are hereby incorporated by reference in their entirety), adenoviral vectors, herpes virus vectors (Fink et al., 1996, Ann. Rev. Neurosci. 19:265-287), packaged amplicons (Federoff et al., 1992, Proc. Natl. Acad. Sci. USA 89:1636-1640, the contents of which are hereby incorporated by reference in their entirety), papilloma virus vectors, picornavirus vectors, polyoma virus vectors, retroviral vectors, SV40 viral vectors, vaccinia virus vectors, and other vectors. Additionally, the vector can also include other genetic elements, such as, for example, genes encoding a selectable marker (e.g., ?-gal or a marker conferring resistance to a toxin), a pharmacologically active protein, a transcription factor, or other biologically active substance.

Methods for manipulating a vector comprising an isolated nucleic acid are well known in the art (e.g., Sambrook et al., 1989, Molecular Cloning: A Laboratory Manual, 2d edition, Cold Spring Harbor Press, the contents of which are hereby incorporated by reference in their entirety) and include direct cloning, site specific recombination using recombinases, homologous recombination, and other suitable methods of constructing a recombinant vector. In this manner, an expression vector can be constructed such that it can be replicated in any desired cell, expressed in any desired cell, and can even become integrated into the genome of any desired cell.

The expression vector comprising the nucleic acid of interest is introduced into the cells by any means appropriate for the transfer of DNA into cells. Many such methods are well known in the art (e.g., Sambrook et al., supra; see also Watson et al., 1992, Recombinant DNA, Chapter 12, 2d edition, Scientific American Books, the contents of which are hereby incorporated by reference in their entirety). Thus, in the case of prokaryotic cells, vector introduction can be accomplished, for example, by electroporation, transformation, transduction, conjugation, or mobilization. For eukaryotic cells, vectors can be introduced through the use of, for example, electroporation, transfection, infection, DNA coated microprojectiles, or protoplast fusion. Examples of eukaryotic cells into which the expression vector can be introduced include, but are not limited to, ovum, stem cells, blastocytes, and the like.

Cells, into which the nucleic acid has been transferred under the control of an inducible promoter if necessary, can be used as transient transformants. Such cells themselves may then be transferred into a subject for therapeutic benefit therein. Thus, the cells can be transferred to a site in the subject such that the peptide of the invention is expressed therein and secreted therefrom and thus reduces or inhibits, for example, inflammatory processes so that the clinical condition of the subject is improved. Alternatively, particularly in the case of cells to which the vector has been added in vitro, the cells can first be subjected to several rounds of clonal selection (facilitated usually by the use of a selectable marker sequence in the vector) to select for stable transformants. Such stable transformants are then transferred to a subject, preferably a human, for therapeutic benefit therein.

Within the cells, the nucleic acid encoding the polypeptide of the present invention is expressed, and optionally is secreted. Successful expression of the nucleic acid can be assessed using standard molecular biology techniques (e.g., Northern hybridization, Western blotting, immunoprecipitation, enzyme immunoassay, etc.).

### Pharmaceutical Compositions of the Invention

The present invention also provides pharmaceutical compositions that can be administered to a subject to achieve a therapeutic effect. Pharmaceutical compositions of this invention can be prepared for administration by combining a polynucleotide or protein of the present invention, having the desired degree of purity in a pharmaceutically effective amount, with pharmaceutically acceptable carriers.

The pharmaceutical compositions of the present invention can be formulated for parenteral (e.g., intravenous, intramuscular and subcutaneous), topical, oral, inhalable, or local administration.

In preparing the compositions in oral liquid dosage forms (e.g., suspensions, elixirs and solutions), typical pharmaceutical media, such as water, glycols, oils, alcohols, flavoring agents, preservatives, coloring agents and the like can be employed. Similarly, when preparing oral solid dosage forms (e.g., powders, tablets and capsules), carriers such as starches, sugars, diluents, granulating agents, lubricants, binders, disintegrating agents and the like will be employed. For topical administration, the compositions of the present invention may be formulated using bland, moisturizing bases, such as ointments or creams. Examples of suitable ointment bases are petrolatum, petrolatum plus volatile silicones, lanolin and water in oil emulsions.

For administration by inhalation, the active agents of the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

The polynucleotides and polypeptides of the present invention are particularly useful for intravenous administration. The compositions for administration will commonly comprise a solution of the polynucleotide or polypeptide dissolved in a pharmaceutical acceptable carrier, preferably in an aqueous carrier. A variety of aqueous carriers can be used, e.g., buffered saline, water, or any physiologically buffered solution designed for intravenous administration. These solutions are sterile and generally free of undesirable matter. The compositions may be sterilized by conventional, well-known sterilization techniques. A typical pharmaceutical composition for intravenous administration can be readily determined by one of ordinary skill in the art. The amounts administered are clearly protein specific and depend on its potency and pharmacokinetic profile. Actual methods for preparing parenterally administrable compositions will be known or apparent to those skilled in the art and are described in more detail in such publications as Remington's Pharmaceutical Science, 18t ed., Mack Publishing Company, Easton, Pa., 1990.

### Uses of the Compositions

The present invention provides methods for inhibiting tumor progression or tumor metastasis in a subject comprising administering to a subject in need of such treatment a pharmaceutical composition comprising a therapeutically effective amount of an antisense polynucleotide.

A “therapeutically effective amount” of an active agent according to the present invention is that amount of the active agent which is sufficient to provide a beneficial effect to the subject to which the active agent is administered. More specifically, a therapeutically effective amount means an amount of the active agent effective to prevent, alleviate or ameliorate tissue damage or symptoms of a disease of the subject being treated.

According to present invention, the subject to be treated with the pharmaceutical compositions is preferably a mammal. According to other embodiments, the mammal is a human.

The tumor to be treated is selected from the group consisting of ovarian cancer, lung cancer, breast cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, and kidney cancer. Particular types of tumors amenable to treatment include: hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell lung carcinoma, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma. Additional types of tumors include non-solid lymphoproliferative disorders and hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

The present invention further provides methods for treating endothelial cell related conditions or disorders which require improved or enhanced lymphatic clearance and/or lymphangiogenesis, the methods comprise administering to a subject in need of such treatment a therapeutically effective amount of an active agent capable of increasing the level of LEDGF/p75 protein.

The terms “treating” or “treatment” refer to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

According to one embodiment, an endothelial cell related condition is inflammation.

Treatment or protection against inflammation may be accomplished in the fetus, newborn, child, adolescent as well as in adults and old persons, whether the inflammation to be treated is spontaneous, chronic, of traumatic etiology, a congenital defect or a teratogenic phenomenon.

According to some embodiments, the inflammation is associated with an inflammatory disease.

According to other embodiments, the inflammatory disease is selected from the group consisting of chronic inflammatory disease and acute inflammatory disease.

According to some embodiments, the inflammatory disease is selected from the group consisting of systemic lupus erythematosus (SLE), inflammatory bowel disease (Crohn's disease), psoriasis, chronic bronchitis, and sepsis.

According to further embodiments, the inflammation to be treated by the pharmaceutical compositions of the invention is associated with autoimmune disease. According to other embodiments, the autoimmune disease is selected from the group consisting of cardiovascular disease, rheumatoid disease, glandular disease, gastrointestinal disease, cutaneous disease, hepatic disease, neurological disease, muscular disease, nephric disease, disease related to reproduction, connective tissue disease and systemic disease.

According to other embodiments, the inflammation to be treated is associated with an injury. According to some embodiments, the injury is selected from the group consisting of an abrasion, a bruise, a cut, a puncture wound, a laceration, an impact wound, a concussion, a contusion, a thermal burn, frostbite, a chemical burn, a sunburn, a desiccation, a radiation burn, a radioactivity burn, smoke inhalation, a torn muscle, a pulled muscle, a torn tendon, a pulled tendon, a pulled ligament, a torn ligament, a hyperextension, a torn cartilage, a bone fracture, a pinched nerve and a gunshot wound.

According to some embodiments, the inflammation to be treated by the pharmaceutical compositions of the invention is associated with an infectious disease. According to additional embodiments, the infectious disease is selected from the group consisting of chronic infectious disease, subacute infectious disease, acute infectious disease, viral disease, bacterial disease, protozoan disease, parasitic disease, fungal disease, mycoplasma disease and prion disease.

According to further embodiments, the inflammation to be treated is associated with a disease associated with transplantation of a graft. The disease associated with transplantation of a graft is selected from the group consisting of graft rejection, chronic graft rejection, subacute graft rejection, hyperacute graft rejection, acute graft rejection and graft versus host disease. According to additional embodiments, the graft is selected from the group consisting of a cellular graft, a tissue graft, an organ graft and an appendage graft.

According to additional embodiments, the inflammation to be treated by the pharmaceutical compositions of the present invention is associated with chronic degenerative neurological disease. The neurological disease include, but not limited to, neurodegenerative disease, multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis and stiff-man syndrome.

According to additional embodiments, the inflammation to be treated by the pharmaceutical compositions of the invention is associated with hypersensitivity. According to further embodiments, the hypersensitivity is selected from the group consisting of immediate hypersensitivity, antibody mediated hypersensitivity, immune complex mediated hypersensitivity, T lymphocyte mediated hypersensitivity and delayed type hypersensitivity.

According to other embodiments, the inflammation to be treated is associated with an allergic disease. According to some embodiments, the allergic disease is selected from the group consisting of asthma, hives, urticaria, pollen allergy, dust mite allergy, venom allergy, cosmetics allergy, latex allergy, chemical allergy, drug allergy, insect bite allergy, animal dander allergy, plant allergy and food allergy.

According to another embodiment, the inflammation is associated with septic shock.

According to further embodiment, the inflammation to be treated is associated with anaphylactic shock.

According to yet further embodiment, the inflammation to be treated is associated with toxic shock syndrome.

According to additional embodiments, the inflammation to be treated is associated with a prosthetic implant. According to some embodiments, the prosthetic implant is selected from the group consisting of a breast implant, a silicone implant, a dental implant, a penile implant, a cardiac implant, an artificial joint, a bone fracture repair device, a bone replacement implant, a drug delivery implant, a catheter, a pacemaker and a respirator tube.

According to further embodiments, the inflammation to be treated is a musculo-skeletal inflammation. According to some embodiments, the musculo-skeletal inflammation is selected from the group consisting of arthritis, muscle inflammation, myositis, a tendon inflammation, tendinitis, a ligament inflammation, a cartilage inflammation, a joint inflammation, a synovial inflammation, carpal tunnel syndrome and a bone inflammation.

As is well known in the art, induction of lymphangiogenesis by induction of expression of vascular endothelial growth factor (VEGF)-C induces healthy tissue homeostasis and is therapeutic for edema, as disclosed, for example, in Cheung L et al (An experimental model for the study of lymphedema and its response to therapeutic lymphangiogenesis. BioDrugs 20(6):363-70, 2006).

As exemplified herein below LEDGF/p75 protein activates VEGF-C expression, and therefore induces lymphangiogenesis. Based on the findings of the present invention, it will now be possible and feasible to administer compounds and compositions that activate expression and activity of LEDGF/p75 for treating edema.

Preferably, the edema that is treated by methods and compositions of the present invention is a lymphedema. More preferably, the edema results from lymphatic vascular insufficiency. In another embodiment, the edema is a tissue edema.

The polynucleotides or polypeptides of the invention can be administered alone or in conjunction with other therapeutic modalities. Thus, it is appropriate to administer the polynucleotides or polypeptides of the invention as part of a treatment regimen involving other therapies, such as surgery and/or drug therapy.

The pharmaceutical compositions of the present invention are administered in a therapeutically effective amount, which will vary depending upon a variety of factors including the activity of the particular active agent employed; the metabolic stability of the active agent; the age, body weight, general health, sex, and diet of the subject; the mode of administration; and the severity of the particular disease.

Having now generally described the invention, it will be more readily understood through reference to the following examples, which are provided by way of illustration and are not intended to be limiting of the present invention.

### EXAMPLES

### Materials and Experimental Methods

### Cell Cultures and Reagents

Cells were cultured in MEM (A549, C6 and MLS), in DMEM (COS7, ES2 and SKOV-3) or RPMI medium (H1299) supplemented with 10% FBS, penicillin and streptomycin. Cells were maintained in 5% CO₂/95% air at 37° C.

### RT-PCR

Total RNA was extracted from cells using Tri Reagent (Molecular Research Center). Two micrograms of total RNA were used for first-strand DNA synthesis using SuperScript II RNase H-reverse (Invitrogen). PCR was performed with the following forward and reverse primers: human VEGF-C (accession no. NM_{—}005429, 5′-CTGCTCGCCGCTGCGCTG [SEQ ID NO: 25] and 5′-GTGCTGGTGTTCATGCACTGCAG [SEQ ID NO: 26]), human LEDGF/p75 (accession no. AF063020, 5′-CACACAGAGATGATTACTACACTG [SEQ ID NO: 27] and 5′-CCATCTTGAGCATCAGATCCTC [SEQ ID NO: 28]), mouse and rat LEDGF antisense (LEDGFas) (accession no. AK042735 and CB576984 5′-CCTGTTGGTTCCTTCTCTAGC [SEQ ID NO: 29] and 5′-GGCGGTTCAAAGTCAGTCAAG [SEQ ID NO: 30]), human LEDGFas (accession no. AV716383 5′-CCTGTTTGTTCCTTCTCTAGC [SEQ ID NO: 31] and 5′-GGCGATTCAAAGTTAGTCAGG [SEQ ID NO: 32]) and human GAPDH (accession no. BC004109, 5′-CGGAGTCAACGGATTTGGTCGTAT [SEQ ID NO: 33] and 5′-AGCCTTCTCCATGGTGGTGAAGAC [SEQ ID NO: 34]). All PCR conditions and primers were optimized to produce a single product of the correct base pair size in the linear range of the reaction. The target mRNA expression level was calculated as the ratio of the target mRNA to GAPDH mRNA for each sample.

### Expression Vectors and Stable Transfections

LEDGF and LEDGFas sequences were reverse transcribed from H1299 mRNA and PCR amplified using Phusion™ high-fidelity DNA polymerase (Finnzymes) together with the following forward and reverse primers:

LEDGF:

| | |
|---|---|
| 5′-ATGACTCGCGATTTCAAACCTGG | (SEQ ID NO: 35) |
| and | |
| 5′-CTAGTTATCTAGGGTAGACTCCTTCAG; | (SEQ ID NO: 36) |
| LEDGFas: | |
| 5′-CCTGTTTGTTCCTTCTCTAGC | (SEQ ID NO: 37) |
| and | |
| 5′-GGCGATTCAAAGTTAGTCAGG. | (SEQ ID NO: 38) |

Fragments were ligated into pCR-BluntII-TOPO (Invitrogen) and their fidelity was confirmed by DNA sequence analysis. Inserts were restricted and ligated into pIRES-Luc or pIRES expression vectors (Hobbs et al, Development of a bicistronic vector driven by the human polypeptide chain elongation factor 1alpha promoter for creation of stable mammalian cell lines that express very high levels of recombinant proteins. Biochem Biophys Res Comm 252:368-372, 1998). Stable transfections were carried out with Lipofectamine 2000™ reagent (Invitrogen), and 2.5 mg/ml puromycin (Sigma) was added 48 h postransfection to initiate selection.

### Luciferase Reporter Assays

VEGF-C sequences (GenBank accession No. NM_{—}005429) was amplified from human genomic DNA by PCR using forward (5′-CCGCCGCAGCGCCCGCG; SEQ ID NO: 39) and reverse (5′-GAGAAGAAGCCCAGCAAGTG; SEQ ID NO: 40) primers with BamHI and XhoI restriction sites respectively. The product was digested and ligated into pLuc, which encodes firefly luciferase. The fidelity of the insert was confirmed by DNA sequence analysis. Mutations were introduced into STRE with in the VEGF-C gene by using mutated PCR primers (ml; 5′-CACTTCGGGGAAGAAAAGGGAGGAGGGGG; SEQ ID NO: 41 and m2; 5′-GCCAGAGCCCTCGTTTTTCTCCTTTCTTTTCTTCCCCGAAGTGAGAG; SEQ ID NO: 42). Twenty-four hours before transfection, cells were plated in a 24-well plate (1×10⁵ per well) and transfected using Lipofectamine 2000™ reagent (Invitrogen) with pSV-Renilla (40 ng), luciferase reporter (300 ng), and 500 ng of pIRES alone, pIRES encoding LEDGF/p75, or pIRES encoding LEDGFas. Twenty-four hours post transfection, luciferase assay was performed (dual luciferase reporter assay system, Promega). Firefly luciferase activity was normalized to Renilla luciferase activity for each transfected well.

### ChIP Assays

Untreated H1299 cells, induced with 0.2 mM H₂O₂ or heated at 42° C. for 1 and 4 hours were fixed in 1% formaldehyde for 10 min. Chromatin Immunoprecipitation Assay was performed with the EZ ChIP Chromatin Immunoprecipitation Kit (Upstate) with anti-LEDGF/p75 antibodies (C16, Santa Cruz). PCR was performed using primers to amplify the 468 by DNA (SEQ ID NO: 43) sequences from the VEGF-C gene (for details see luciferase reporter assay section).

### Immunoblot Assays

Whole-cell lysates were prepared in ice-cold RIPA buffer (20 mM Tris, pH 7.4, 137 mM NaCl, 10% glycerol, 0.5% (wt/vol) sodium deoxycholate, 0.1% (wt/vol) sodium dodecyl sulfate (SDS), 1% Triton X-100, 2 mM EDTA) containing 1 mM phenylmethylsulfonyl fluoride (PMSF) and protease inhibitor cocktail (Sigma) and fractionated by SDS-PAGE. Primary antibodies against the following proteins were used: VEGF-C(C-20, Santa Cruz), LEDGF/p75 (C16, Santa Cruz) and b-tubulin (Santa Cruz). HRP-conjugated anti-rabbit secondary antibodies (Jackson ImmunoResearch Laboratories) were used.

### Tumor Xenografts

A pool of cloned H1299 cells (5×10⁶ in 100 microliter [mcl] PBS) expressing luciferase either alone (pIRES-Luc) or together with LEDGF (pIRES-Luc-LEDGF) were subcutaneously inoculated into the hind limb of six-week-old CD-1 nude female mice. Ten days post-inoculation mice were injected with 150 mg D-luciferin (Xenogen) and subjected to luciferase bioluminescence imaging (IVIS 100; Xenogen). Two weeks later tumors were removed and their diameter was measured by a caliber. Tumors were fixed (overnight; 4% paraformaldehyde in DEPC-PBS) and then embedded in paraffin blocks.

### Histology

Fixed paraffin-embedded tumor blocks were sectioned serially. The first slide was stained with haematoxylin and eosin (H&E), while other representative slides underwent immunohistochemical staining and in situ hybridization. Unstained sections were deparaffinized with xylene for 5 min followed by sequential ethanol hydration and double-distilled water. Sections were then washed with PBS for 5 min, blocked by overnight incubation in 1% BSA in PBS at 4° C., and stained with monoclonal anti-smooth muscle actin (SMA; Sigma; stain for pericytes and vascular smooth muscle cells), conjugated to alkaline phosphatase. HA-LEDGF expression was determined using Alexa Fluor-labeled antibody to HA (Covance; HA.11 monoclonal antibody). To visualize lymphatic endothelial cells, staining was carried out with LYVE-1 antibodies (Fitgerald Industries). Apoptosis was assessed by terminal deoxynucleotidyl transferase-mediated deoxyuridine triphosphate-biotin nick-end labelling (TUNEL; ApopTag Plus Peroxidase in situ Apoptosis Detection Kit; Chemicon, Temecula, Calif., USA).

### In Situ Hybridization

A probe specific for VEGF-C coding region designed for in situ hybridization was prepared by RT-PCR using forward (5′-CTGTGTCCAGCGTAGATGAGC; SEQ ID NO: 44) and reverse (5′-GTAGACGGACACACATGGAGG; SEQ ID NO: 45) primers. This probe was aimed to share high degree of homology with human and mouse cDNAs (accession number NM_{—}005429 and NM_{—}009506 respectively). Sequence was verified and the fragment (282 bp) was cloned into the pGEM-T Easy vector system (Promega). Digoxigenin-labeled riboprobes were produced by in vitro transcription using a digoxigenin RNA labeling kit (Roche). Paraffin sections of H1299 tumors were deparaffinized, and then proteinase K (Sigma) digestion was carried out, followed by postfixation in 4% paraformaldehyde in PBS. After 2 TBS rinses, sections were dehydrated and air-dried. Slides were preincubated with hybridization mixture [2×standard saline citrate (SSC), 10% dextran sulfate, 1×Denhardt's solution (Sigma), 50% formamide and 0.02% SDS] in a humidified oven for 30 min at 55° C. Hybridization was initialized by addition of digoxigenin-labeled antisense or sense riboprobes (1 mg/ml), yeast tRNA (100 mg/ml, Sigma) and carried out overnight under the above conditions. At the end of incubation, slides were rinsed (2×10 min, RT) in 2×SSC containing EDTA (1 mM), once (1 hr, 50° C.) in 0.2×SSC containing EDTA, twice (10 min, RT) in 0.5×SSC, TBS for 5 min, RT, and TBS containing BSA (1%; Sigma) for 1 hr at room temperature. Slides were incubated with antidigoxigenin alkaline phosphatase (Roche) and developed using BCIP/NTB substrate kit for histochemistry (Roche).

### Cell Migration and Invasion Assay

H1299 cells (3×10⁵ cells per well) expressing luciferase alone or together with LEDGF/p75 were plated in the upper compartment of a 24-well Transwell tray (Corning, Acton, Mass.) for cell migration or in a Matrigel™ Invasion Chamber (BD BioCoat) for cell invasion. Cells were plated in 1% FBS-containing medium while the lower compartment contained full medium. Cells were allowed to migrate/invase through the intervening nitrocellulose membrane (8 mm pore size with or without Matrigel™) during 16 h of incubation at 37° C. Inserts were removed, fixed for 15 min in phosphate-buffered saline (PBS) containing paraformaldehyde (4%), and stained with methyl violet. Cells growing on the upper side of the filter were scraped using cotton swabs, whereas cells growing on the bottom side of the filter were photographed and counted using ImageJ.

### Bioinformatics Analyses

Transcription factor binding site prediction was performed with TFSearch Version 1.3: (Heinemeyer T et al, Databases on Transcriptional Regulation: TRANSFAC, TRRD, and COMPEL. Nucl Acids Res 26: 364-370, 1998). Genomic analyses were performed at the UCSC Genome Browser (Kent, W J et al. The human genome browser at UCSC. Genome research 12: 996-1006, 2002). Genome builds used were: Mouse—mm8; Human—hg18; Rat—rn4; Cow—BosTau2. Tracks used (from the various genomes) include: mRNAs; Spliced ESTs; ESTs; Human, Mouse, Cow and Rat Nets; and Conservation.

### Statistical Analysis

Data is presented as average±SD of at least three independent experiments. Statistical significance (p<0.05) was assessed by t test.

### Example 1

### Co-Expression of VEGF-C and LEDGF in Cancer Cells

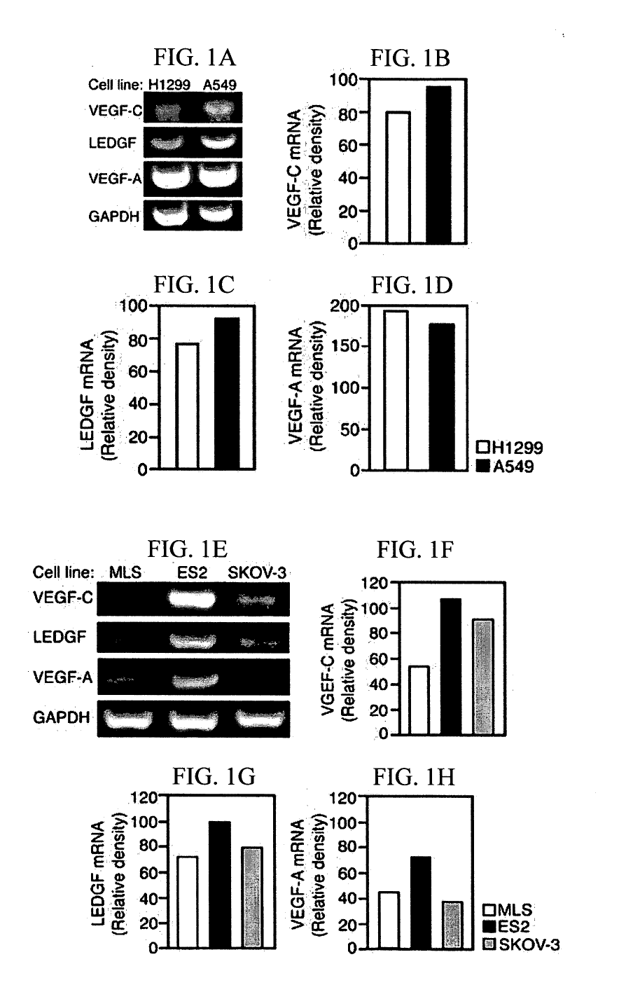

Expression of VEGF-C, VEGF-A, and LEDGF/p75, survival factors regulated by environmental stresses, was compared by RT-PCR in various cancer cell lines including two human lung cancer cell lines, namely H1299 and A549, non-small lung and type II alveolar carcinoma, respectively (FIG. 1A). mRNA expression levels were normalized against GAPDH (FIG. 1B-D respectively). Expression of VEGF-C, VEGF-A, and LEDGF/p75 was compared by RT-PCR in human ovarian (MLS, ES2 and SKOV-3, FIG. 1E) cell lines. mRNA expression levels were normalized against GAPDH (FIG. 1F-H respectively).

Example 1 shows that LEDGF/p75 expression is correlated with that of VEGF-C, but not VEGF-A.

### Example 2

### Over-Expression of LEDGF/p75 Triggers Transcription of VEGF-C mRNA

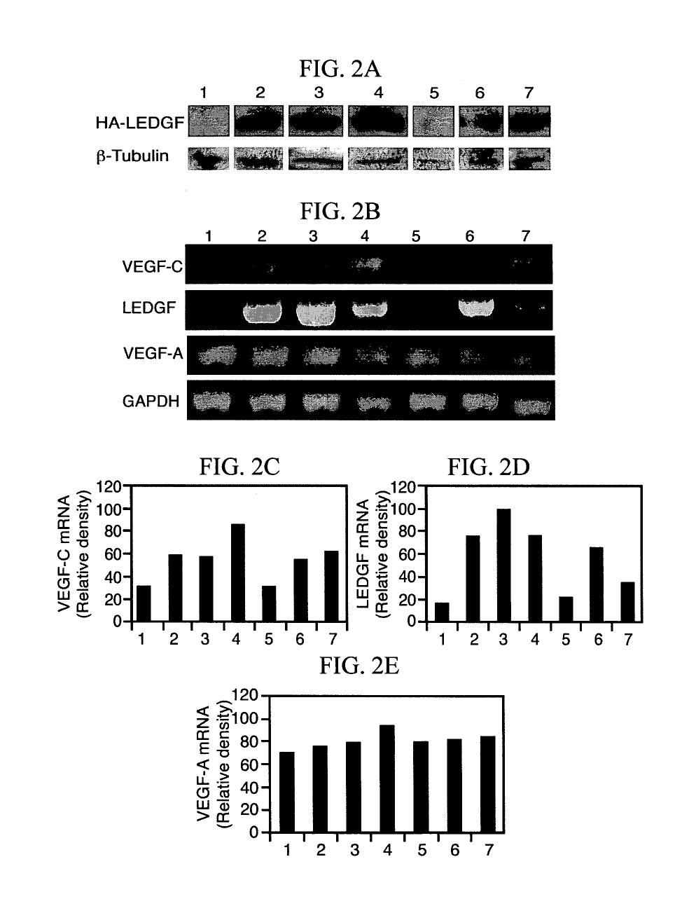

H1299 cells were transfected either with a luciferase expression vector (pIRES-Luc) or with a construct encoding both luciferase and rat LEDGF tagged with human influenza virus hemagglutinin (HA) epitope (pIRES-Luc-LEDGF). Several puromycin-resistant pools of cells as well as individual clones were selected, and expression level of LEDGF/p75 was evaluated by anti-HA Western blot analysis. A strong correlation between LEDGF/p75 and VEGF-C mRNA expression level was detected both in the pool of cells (FIG. 2A-E lanes 1-2; regression analysis for all cell lines, p=0.007), and in the individual clones as determined by RT-PCR (FIG. 2A-E lanes 3-7) and confirmed by quantification of the relative signals, while no correlation was observed between expression level of VEGF-A and either LEDGF/p75 or VEGF-C (regression analysis of all cell lines, p=0.35 and 0.7 respectively).

Example 2 shows that expression of LEDGF/p75 is sufficient to induce expression of VEGF-C, but not VEGF-A, in cancer cell lines.

### Example 3

### Identification of LEDGF/p75 Binding Sites in the VEGF-C Gene

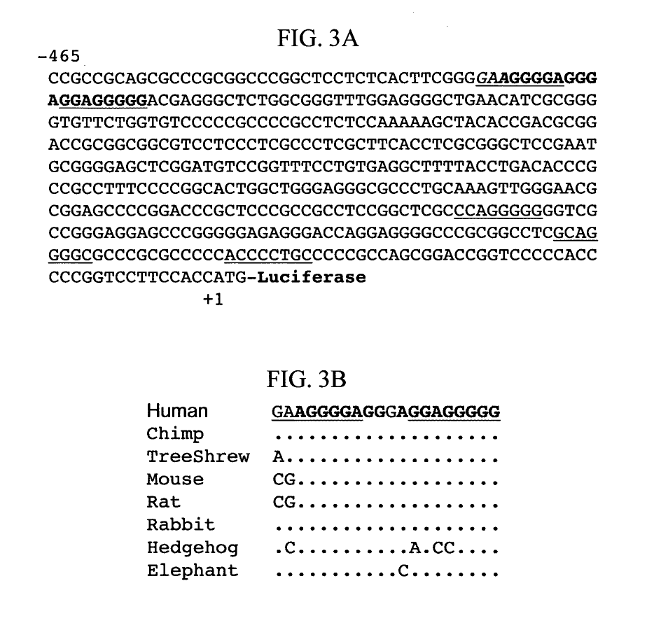

To evaluate the molecular basis of the correlation between the mRNA expression levels of VEGF-C and LEDGF/p75, the proximal region of the human VEGF-C promoter (chr4:177,950,457-177,950,924, UCSC build hg18) was searched for matches to the known consensus LEDGF/p75 binding sites (STRE). Five candidate STREs were identified in a 468 by 5′-flanking region (as set forth in SEQ ID NO: 43) of the human VEGF-C gene, including the 5′ UTR and the region directly upstream, (FIG. 3A), of which two gaagggga [SEQ ID NO: 46] and ggaggggg [SEQ ID NO: 47] were found to be highly conserved between various mammalian species (FIG. 3B); these two STREs were juxtaposed to a putative HSE binding site and separated by two AGG head-to-head repeat boxes, consistent with STRE function, forming the combined sequence gaaggggagggaggaggggg (SEQ ID NO: 48).

Example 3 shows LEDGF/p75 binding sites in human VEGF-C promoter.

### Example 4

### LEDGF/p75 Induces VEGF-C Promoter Activity

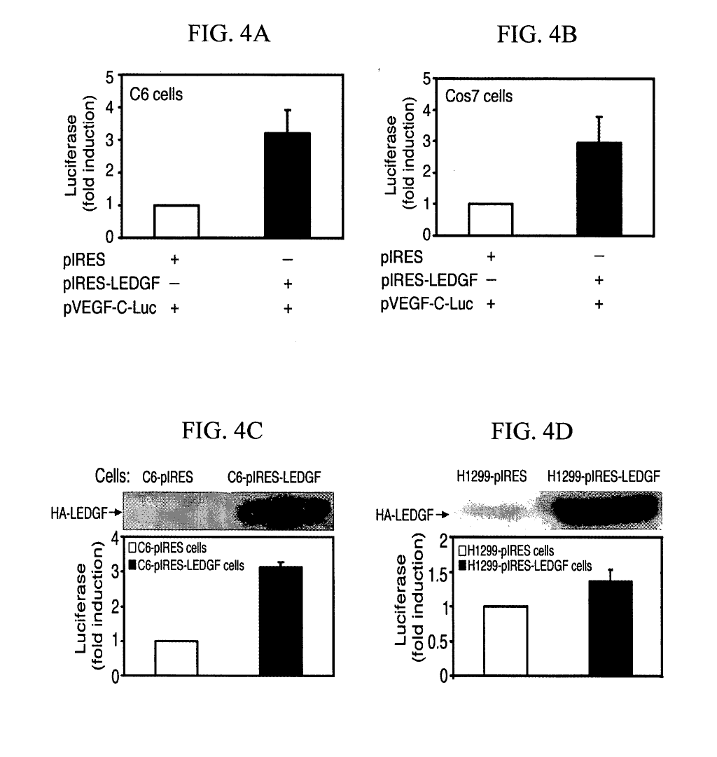

To determine whether the 5′-flanking region of the human VEGF-C gene could mediate a transcriptional response to LEDGF/p75, the 468 by DNA fragment spanning the STREs (SEQ ID NO:46-48) was inserted into a promoter-less luciferase reporter plasmid to generate the reporter construct pVEGF-C-Luc. Rat glioma C6 (FIG. 4A) and COS7 (FIG. 4B) were transiently cotransfected with pVEGF-C-Luc either together with a LEDGF/p75-encoding construct (pIRES-LEDGF) or a control empty vector (pIRES). 48 hours post-transfection, VEGF-C promoter activity was induced as much as four-fold in cells expressing LEDGF/p75. Similar results were observed in C6 and H1299 cells stably over-expressing LEDGF/p75 (FIGS. 4C and 4D, respectively).

Example 4 shows LEDGF/p75 induces VEGF-C promoter activity in various cell lines including glioma and lung cancer.

### Example 5

### LEDGF/p75 Activates the VEGF-C Promoter by Interaction with at Least the Distal of the Two Conserved STRE Sequences

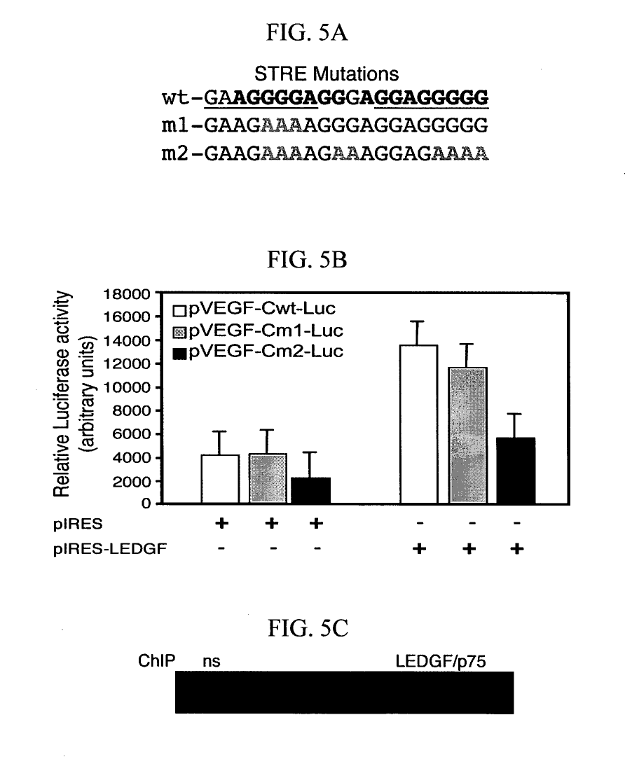

To determine the importance of the conserved STREs for VEGF-C promoter activity, several G to A substitutions were introduced into these sites (FIG. 5A), thus inactivating either the proximal putative STRE (pVEGF-Cm1) or both STRE sequences and one AGG box (pVEGF-Cm2). Promoter activity was measured in the absence or presence of LEDGF/p75 expression, in comparison with the intact promoter construct. pVEGF-Cm1-Luc exhibited a modest 15% loss of activity, which was observed only in the presence of LEDGF/p75. By contrast, pVEGF-Cm2-Luc exhibited a 45% and 68% loss of VEGF-Cwt promoter activity in absence or presence, respectively, of LEDGF/p75 (FIG. 5B). Binding of LEDGF/p75 to the VEGF-C promoter within living cells was detected by the chromatin immunoprecipitation (ChIP) assay on H1299 cells, using a specific anti-LEDGF antibody. Nonspecific antibodies served as a negative control (FIG. 5C).

Example 5 shows that LEDGF/p75 selectively transactivates VEGF-C gene transcription by STRE binding.

### Example 6

### VEGF-C Expression is Activated by Oxidative Stress Conditions in a STRE-Dependent Manner

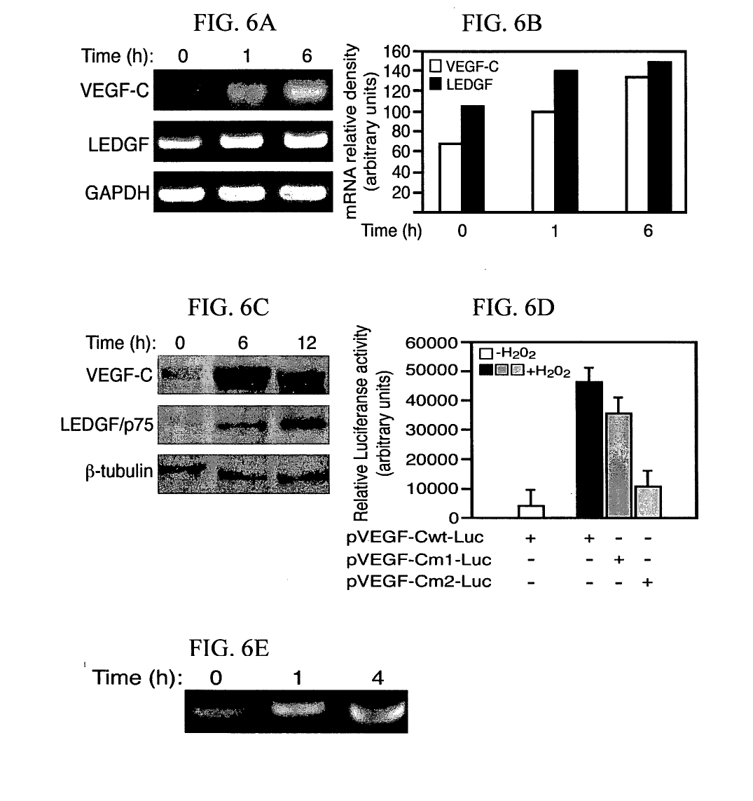

Expression of LEDGF/p75 is activated by micro-environmental stress, resulting in induced expression of LEDGF/p75 target genes (AOP2, αB-crystallin and HSP-27). Regulation of VEGF-C mRNA expression by oxidative stress (0.2 mM H2O2) was evaluated. VEGF-C and LEDGF/p75 mRNA expression and protein levels were co-induced under oxidative stress conditions as early as one hour and reached up to 3 and 1.5 fold induction, respectively, after 6 h, as shown by RT-PCR (FIG. 6A-B) and by immunoblot using a subunit-specific antibody directed to the human VEGF-C precursor or the p75 variant of LEDGF (FIG. 6 C). The role of LEDGF/p75 binding sites in the VEGF-C promoter for oxidative and thermal-induced promoter activity was determined by luciferase assay. H1299 cells were transiently transfected either with intact (pVEGF-Cwt-Luc) or mutated (pVEGF-Cm1-Luc and pVEGF-Cm2-Luc) reporter constructs and subjected to oxidative stress (0.2 mM H₂O₂ for 24 hr), and luciferase activity was measured. pVEGF-Cwt-Luc exhibited a ten-fold increase in expression over un-stimulated cells in response to oxidative stress (FIG. 6D). Disruption of the proximal STRE site and both LEDGF/p75 sites diminished the promoter activity by 23% and 76%, respectively. These results were corroborated by ChIP analysis, which demonstrated that binding of LEDGF/p75 to the VEGF-C promoter was significantly enhanced as early as 1 h post exposure to oxidative stress (FIG. 6E).

Example 6 shows that VEGF-C expression is activated by oxidative stress conditions in a STRE-dependent manner.

### Example 7

### VEGF-C Expression is Activated by Thermal Stress Conditions in a STRE-Dependent Manner

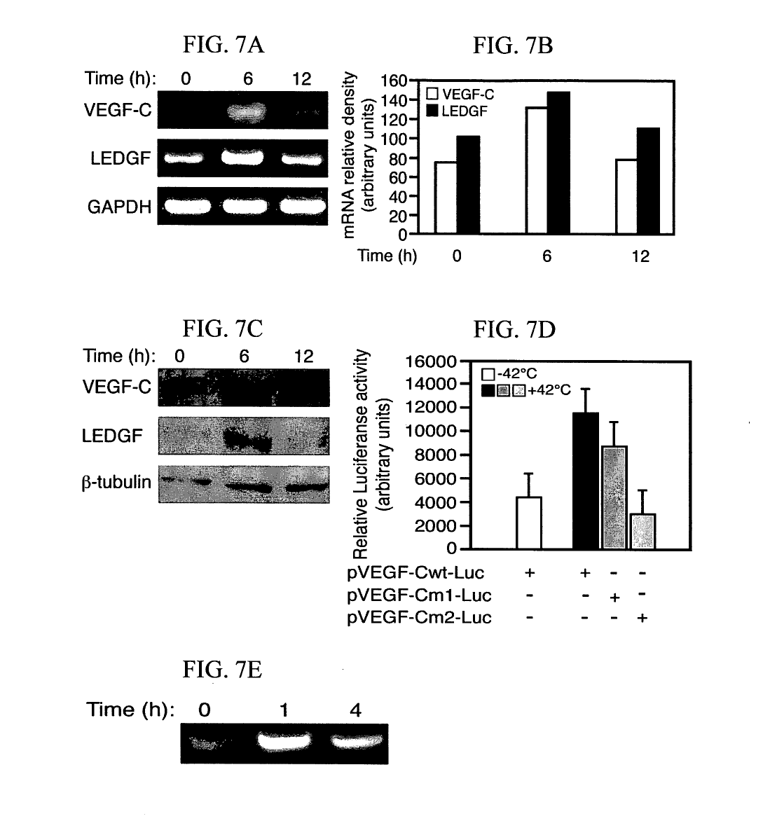

Regulation of VEGF-C mRNA expression by thermal stress was evaluated. Thermal stress (42° C. for 6 hr) resulted in a two-fold enhancement in VEGF-C mRNA (FIG. 7A-B) and protein (FIG. 7C) levels. In addition, thermal stress elicited two-fold increase in VEGF-C promoter activity, which was reduced by 23% and 73% upon disruption of just the proximal or both proximal and distal STRE's, respectively (FIG. 7D). Similar to oxidative stress, ChIP analysis demonstrated enhanced binding of LEDGF/p75 to the VEGF-C promoter as early as 1 h post exposure to thermal stress (FIG. 7E).

Example 7 shows that VEGF-C expression is activated by thermal stress conditions in a STRE-dependent manner.

### Example 8

### Existence of LEDGF/p75 cis-Native Antisense Transcripts (cis-Nat)

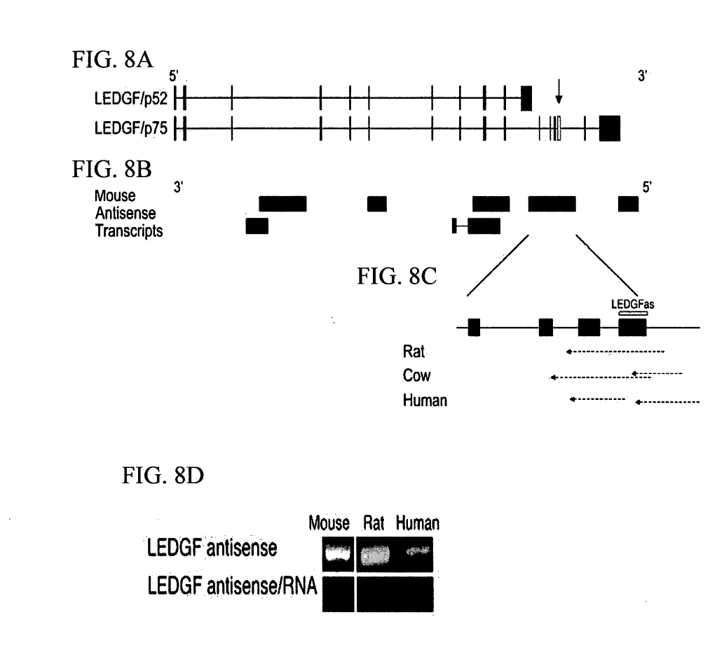

A search of the LEDGF/p75 locus in the mRNA track of the mouse genome database (UCSC genome browser [Kent et al, The human genome browser at UCSC. Genome Res 12: 996-1006, 2002], genome build mm8, FIG. 8A) identified several cDNAs oriented in a direction consistent with transcription from the opposite strand of the LEDGF locus, representing putative cis-encoded natural LEDGF antisense mRNAs. One putative native antisense transcript (NAT), AK042735 (SEQ ID NO:7), was specific to the p75 variant of LEDGF. AK042735 is a 3184 by long single exon transcript and does not appear to encode for a protein. It overlaps exons 11-14 of LEDGF p75 on the opposite strand (FIG. 8B) and completely overlaps the LEDGF p75 locus. Additional database searches identified similar EST clone sequences within the rat (SEQ ID NO:4) and cow (SEQ ID NOs:5-6) EST databases (genome builds: rn4 and bosTau2, respectively, FIG. 8C). Furthermore, in the human database, two discontinuous ESTs were discovered in-silico (genome build hg18; SEQ ID NOs:2-3) by RT-PCR and DNA sequence analysis of contiguous mRNA (FIG. 8C-D). Similarly, existence of mouse and rat putative NATs was verified by RT-PCR, and fidelity of the products was confirmed by DNA sequence analysis (FIG. 8D).
Example 8 shows the existence of LEDGF/p75 cis-NATs.

### Example 9

### LEDGF/p75 cis-NAT Diminishes VEGF-C mRNA Expression

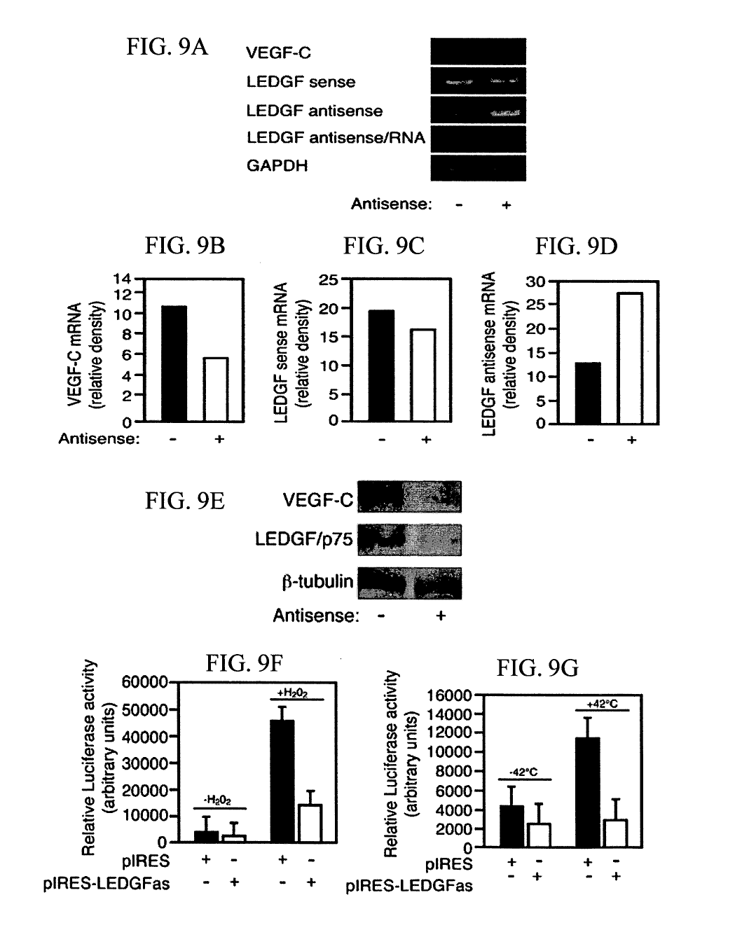

To investigate the ability of LEDGF/p75 antisense RNA to regulate VEGF-C mRNA and protein levels, the human NAT (SEQ ID NO:1) was inserted into the pIRES plasmid vector (pIRES-LEDGFas) and stable transfectants of human lung A549 cells were generated. Expression level of the antisense RNA in a puromycin-selected pool of clones was increased two-fold compared to cells transfected with an empty vector (FIGS. 9A and 9D). In these cells, expression of LEDGF/p75 sense mRNA was reduced by only 15% (FIGS. 9A and 9C), while VEGF-C mRNA levels were strongly reduced (46%, FIGS. 9A and 9B). A robust reduction in protein levels of both LEDGF/p75 and VEGF-C was detected by immunoblot utilizing specific antibodies (FIG. 9E).

Ability of LEDGF/p75 antisense RNA to interfere with stress-induced transcriptional activation of the VEGF-C promoter was tested in H1299 cancer cells transiently cotransfected with pVEGF-C-Luc and pIRES-LEDGFas, with pVEGF-C-Luc+empty pIRES vector as the negative control. Cells were subjected either to oxidative (0.2 mM H₂O₂ for 24 hr) or thermal (42° C. for 6 hr) stress. Both basal and stress-induced promoter activity were significantly attenuated by the presence of LEDGF/p75 antisense RNA expression (FIGS. 9F and 9G).

Example 9 shows that LEDGF/p75 antisense RNA can inhibit both basal and LEDGF/p75-induced VEGF-C expression and activity in response to stress induction.

### Example 10

### VEGF-C Expression is Induced in H1299 Tumors Over-Expressing LEDGF In Vivo

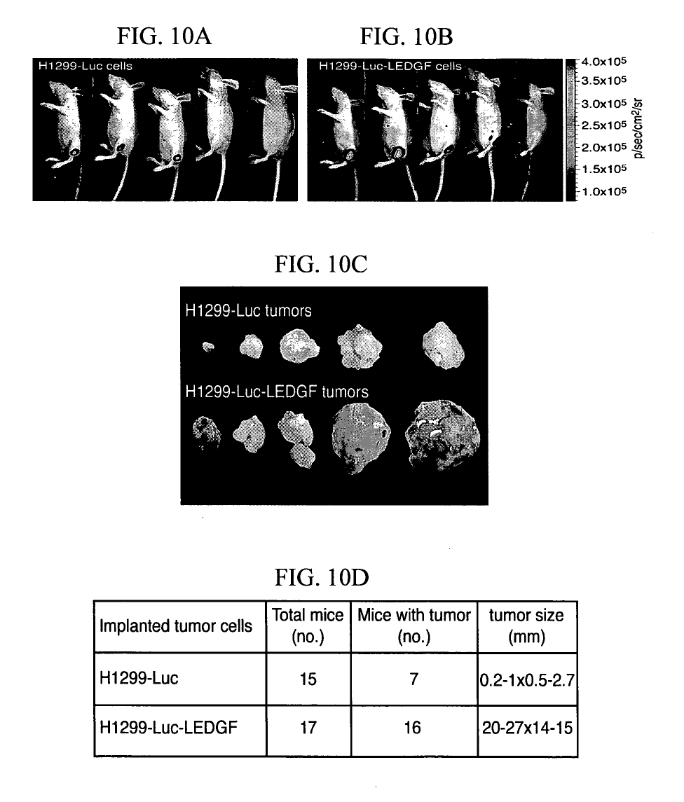

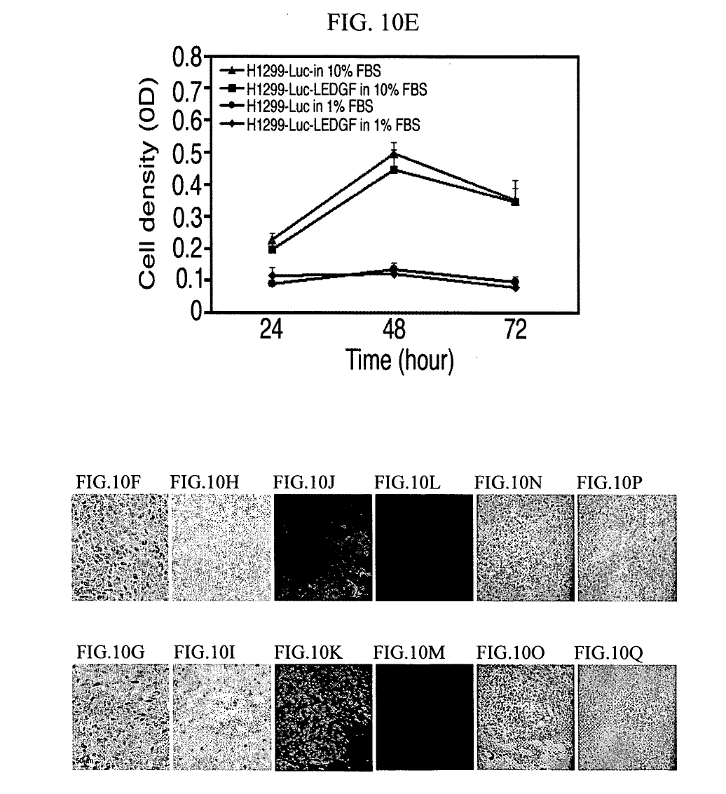

The effect of LEDGF/p75 over-expression on VEGF-C expression, tumor lymphangiogenesis, and tumor progression was studied in subcutaneous tumor xenografts. Pooled H1299 cells (5×10⁶ cells/mouse) either over-expressing the luciferase gene alone (H1299-Luc) or co-expressing luciferase and LEDGF (H1299-Luc-LEDGF) were subcutaneously inoculated into the hind limb of CD-1 nude mice (luciferase activity of preinoculated cells of the two groups was equivalent). Tumor growth was followed in vivo by luciferase bioluminescence imaging. A substantial enhancement in bioluminescence (FIG. 10A-B), corresponding to increased tumor growth, as was evaluated following tumor removal (FIG. 10C-D), was detected in the mice inoculated with H1299-Luc-LEDGF in comparison to the control group. Further, in three independent experiments, 16/17 mice inoculated with H1299-Luc-LEDGF cells, compared with 7/15 inoculated with H1299-Luc, developed tumors. The robust augmentation in tumor growth was not due to a change in cell proliferation rate or viability (FIG. 10E).

The impact of LEDGF/p75 overexpression was evaluated in histological specimens. No pathological differences were observed in histological sections stained with hematoxylin-eosin (FIG. 10E-G). In situ hybridization analysis demonstrated enhanced VEGF-C expression in H1299-Luc-LEDGF tumors (FIG. 10H-I), consistent with the expression level of LEDGF (FIG. 10J-K). LYVE-1, a molecular marker specific for lymphatic endothelial cells, revealed that over-expression of LEDGF/p75 promoted lymphatic vessels formation within and around tumors (FIG. 10L-M). Angiogenesis, on the other hand, was not induced by LEDGF over-expression, as seen by immunohistochemical staining of alpha smooth muscle actin (alpha-SMA; FIG. 10N-O). Similarly, blood volume fraction and vascular permeability measured by dynamic contrast enhanced MRI showed no differences. Despite the difference in tumor growth rate, no alteration in the apoptotic ratio between control and LEDGF overexpressing tumors could be seen utilizing TdT-mediated dUTP-biotin nick end labeling (TUNEL analysis, FIG. 10P-Q).

Example 10 shows that LEDGF induces VEGF-C expression and regulates lymphangiogenesis in tumors.

### Example 11

### LEDGF Enhances Tumor Cell Migration and Invasion

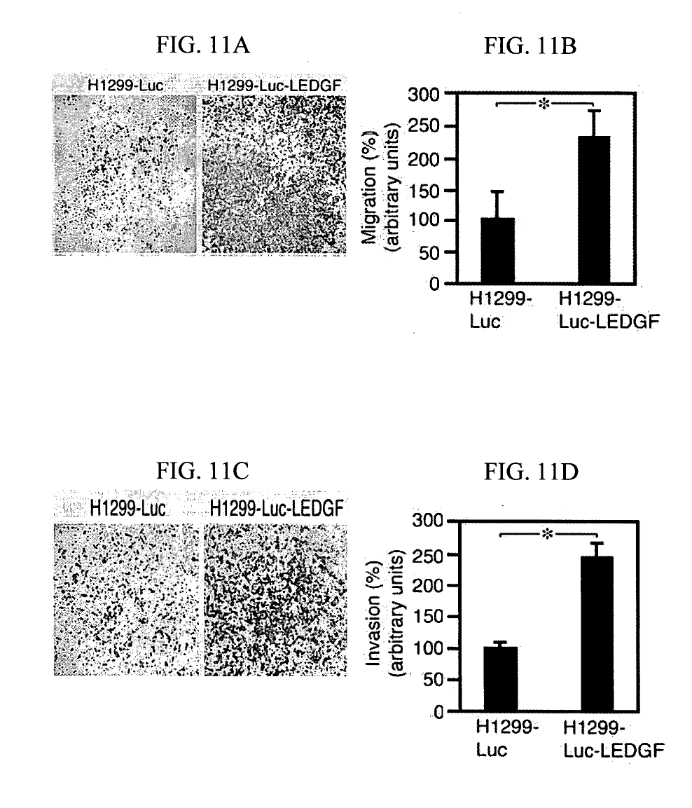

To determine the effect of LEDGF/p75 on tumor cell migration and invasion, transwell migration and Matrigel™ invasion assays were carried out utilizing pools of H1299 cells over-expressing luciferase either alone or together with LEDGF/p75. LEDGF/p75 over-expression caused significant elevation (more than 2.5-fold) of both migration (FIG. 11A-B) and invasion (FIG. 11C-D) of cells compared to H1299-Luc cells.

Example 11 shows that LEDGF/p75 augments tumor progression in vivo by stimulating tumor cell migration and invasion.

### Example 12

### LH Stimulation Induces VEGF-C Expression In Vitro

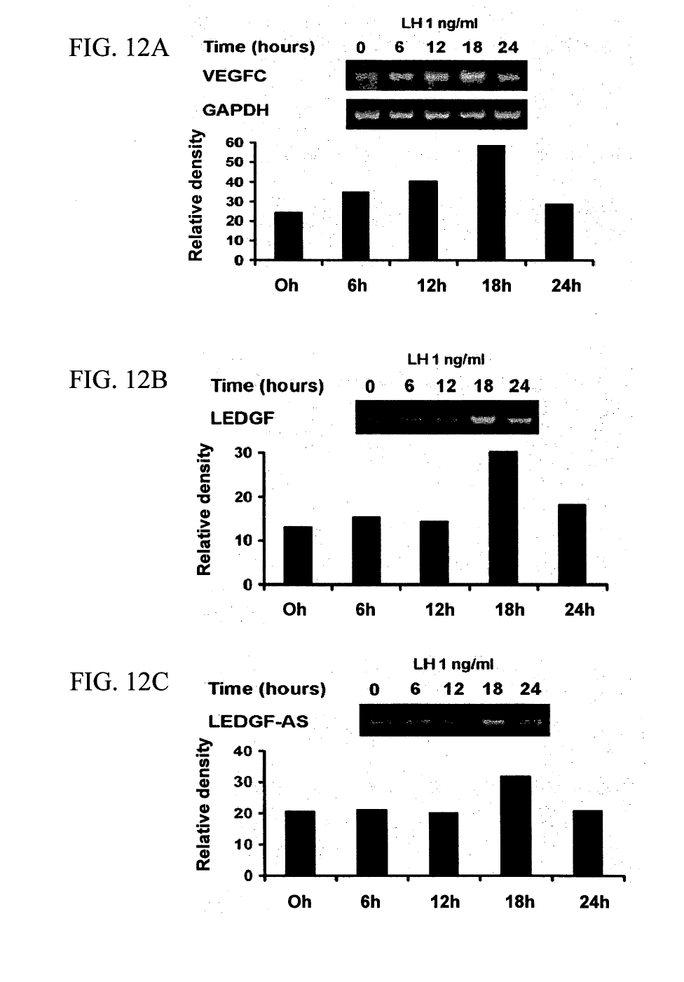

Elevation of gonadotropins levels, typically observed in post-menopausal women, is known to increase the risk for ovarian cancer and to enhance progression of the disease, as well as adhesion and angiogenesis of the tumor. Therefore, the connection between elevation of LH and VEGF-C activation was examined in ES2 ovarian carcinoma cells starved for 24 h in serum free medium. RT-PCR analysis revealed a significant increase in VEGF-C (FIG. 12A) and LEDGF/p75 (FIG. 12B) mRNA levels following LH (1 ng/ml) stimulation. Similar behavior was observed for the LEDGF/p75 antisense transcript (FIG. 12C), suggesting hormonal regulation of LEDGF/p75 antisense on LEDGF/p75 and VEGF-C regulation.

Example 12 shows that LH stimulation induces VEGF-C expression in vitro.

### Example 13

### FSH Stimulation Induces VEGF-C Expression In Vitro

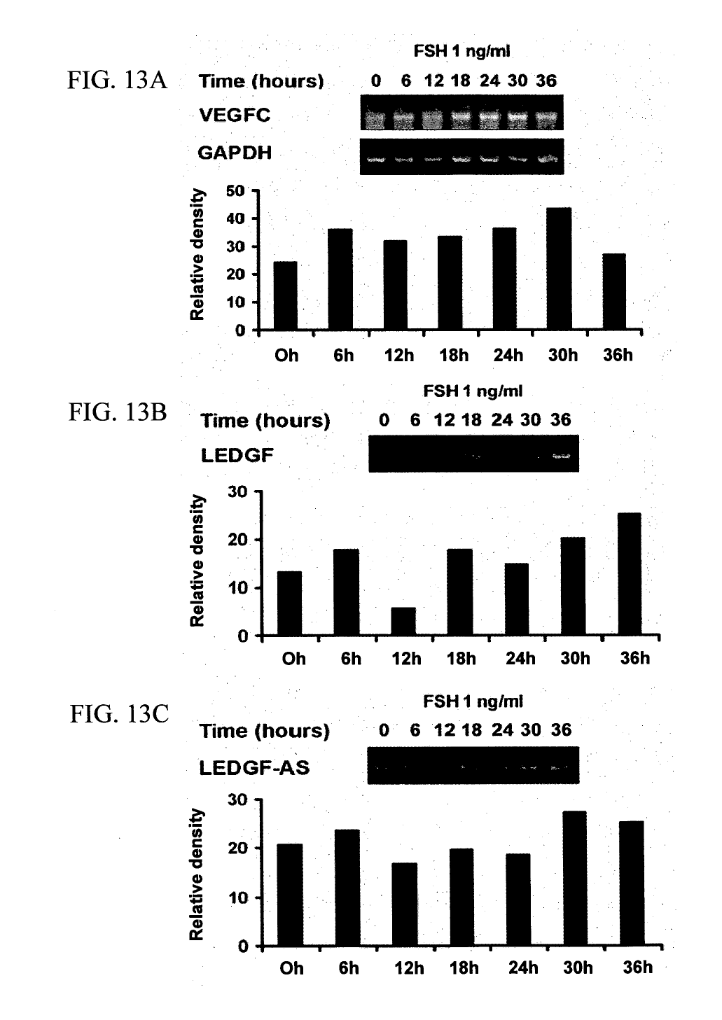

The connection between elevation of FSH and VEGF-C activation was examined in ES2 ovarian carcinoma, as well. Similar to LH stimulation (Example 12), FSH (1 ng/ml) stimulation significantly increased VEGF-C (FIG. 13A), LEDGF/p75 (FIG. 13B) mRNA levels and LEDGF/p75 antisense transcript (FIG. 13C).

Example 13 shows that FSH stimulation induces VEGF-C expression in vitro.

### Example 14

### In Vitro Hormonal Stimulation Induces VEGF-C Activation

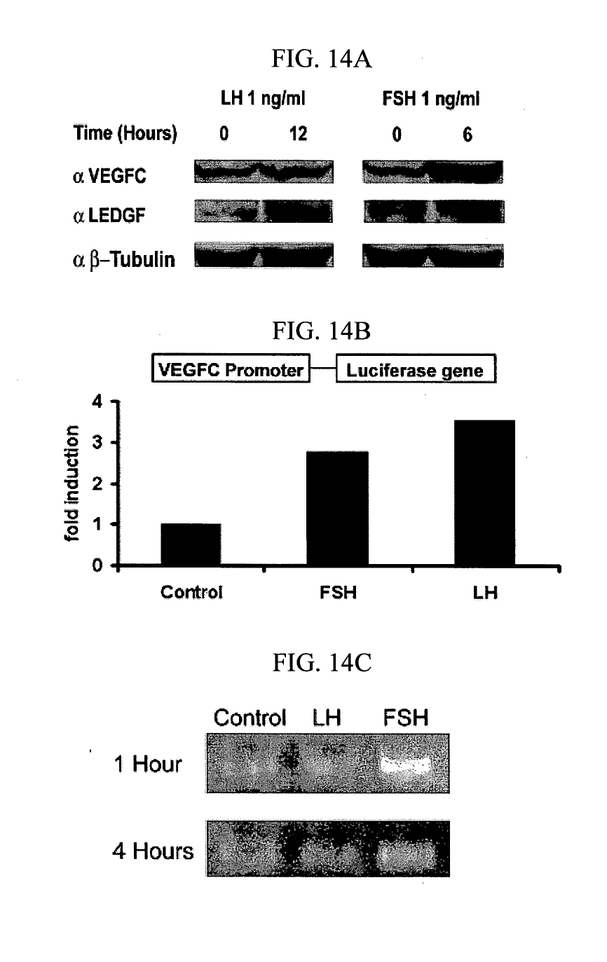

The connection between elevation of LH and FSH, and VEGF-C activation was further examined in ES2 ovarian. Western blot analysis indicated a significant elevation in both LEDGF/p75 and VEGF-C protein levels following treatment (FIG. 14A). The effect of hormonal stimulation on the VEGF-C promoter activation was determined by luciferase assay. ES2 cells were transiently transfected with pVEGF-Cwt-Luc reporter construct and subjected to LH or FSH hormonal stimulation (1 ng/ml for 18 h). Analysis of the luciferase signal revealed up to 3.5 fold increase in the VEGF-C promoter activity compared to unstimulated cells (FIG. 14B). These results were corroborated by ChIP analysis, which demonstrated that binding of LEDGF/p75 to the VEGF-C promoter was significantly enhanced following hormonal stimulation (1 h and 4 h; FIG. 14C).

Example 14 shows that in vitro hormonal stimulation induces VEGF-C activation, probably in a LEDGF/p75 dependent manner.

### Example 15

### Hormonal Stimulation Induces VEGF-C Promoter Activation In Vivo

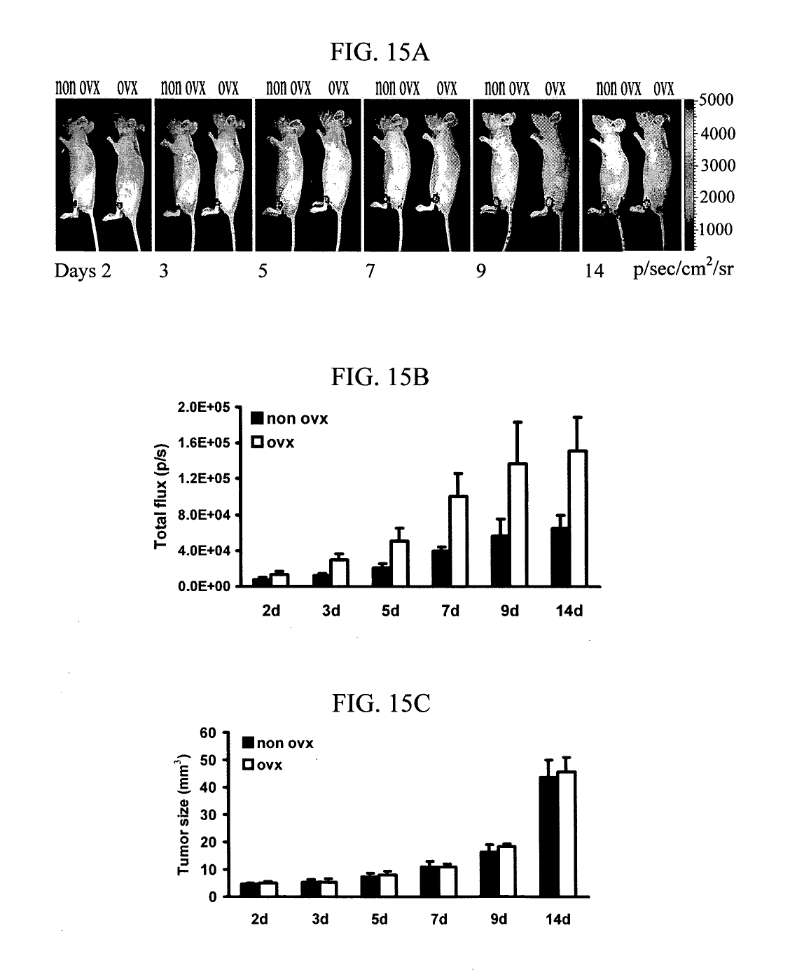

The role of gonadotropins stimulation in VEGF-C activation was studied in a mouse ovariectomy model, known to induce elevation of LH and FSH levels. ES2 cells stably transfected with the pVEGF-Cwt-Luc construct were subcutaneously inoculated into the hind limb of ovariectomy nude mice. Tumor growth was followed in vivo by luciferase bioluminescence imaging. A substantial enhancement in bioluminescence, corresponding to VEGF-C promoter activity was detectable within 2 days of tumor initiation as compared to control unovariectomized female mice (FIG. 15A). Analysis of total flux of photons from the tumor area showed a significant elevation of the signal in the ovariectomized group compared to control (FIG. 15B). Manual analysis of tumor size did not reveal significant differences between the two groups, suggesting that the elevation in the signal is not a mere result of changes in the tumor growth rate (FIG. 15C).

Example 15 shows that VEGF-C is hormonally regulated also in vivo and might influence the physiology of the tumor.

In summary, the findings presented herein demonstrate the role of LEDGF/p75 in controlling a novel stress pathway allowing microenvironment regulation of structural changes in the lymphatic vasculature through expression of VEGF-C. Lymphangiogenesis induced in response to stress cues can alleviate edema and help maintain tissue homeostasis by augmenting the capacity for fluid clearance. Furthermore, induction of lymphangiogenesis can facilitate an innate immune response to danger signals. The effect of tumor progression and lymphangiogenesis suggests that LEDGF/p75 should be evaluated as a potential target for cancer therapy.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1. An antisense polynucleotide complementary to a LEDGF/p75 mRNA comprising the nucleotide sequence as set forth in SEQ ID NO:1 or an active homolog or fragment thereof.

2. The antisense polynucleotide according to claim 1 comprising from about 12 nucleotides to about 700 nucleotides.

3. The antisense polynucleotide according to claim 1 consisting of from about 50 to about 300 nucleotides.

4. The antisense polynucleotide according to claim 1 complementary to a human LEDGF/p75 mRNA, said antisense polynucleotide comprises the nucleotide sequence as set forth in any one of SEQ ID NO:2 and SEQ ID NO:3, or an active homolog or fragment thereof.

5. The antisense polynucleotide according to claim 1 complementary to a human LEDGF/p75 mRNA, said antisense polynucleotide consists of the nucleotide sequence as set forth in SEQ ID NO:1.

6. The antisense polynucleotide according to claim 1 complementary to a non human LEDGF/p75 mRNA, said antisense polynucleotide comprises the nucleotide sequence as set forth in any one of SEQ ID NO:4 to SEQ ID NO:6, or an active homolog or fragment thereof.

7. A pharmaceutical composition comprising as an active agent an antisense polynucleotide complementary to a LEDGF/p75 mRNA, the antisense polynucleotide comprising the nucleotide sequence as set forth in SEQ ID NO:1 or an active homolog or fragment thereof; further comprising a pharmaceutically acceptable carrier.

8.-12. (canceled)

13. A method for inhibiting tumor progression or tumor metastasis in a subject comprising administering to a subject in need of such treatment a pharmaceutical composition comprising a therapeutically effective amount of an antisense polynucleotide according to claim 1.

14. The method according to claim 13, wherein the tumor is selected from the group consisting of ovarian carcinoma, lung carcinoma, breast carcinoma, prostate carcinoma, pancreas carcinoma, liver carcinoma, colon carcinoma, hepatocellular carcinoma, rectal carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma, renal cell carcinoma, hypernephroma, hypernephroid adenocarcinoma, bile duct carcinoma, choriocarcinoma, seminoma, embryonal carcinoma, Wilms' tumor, testicular tumor, bladder carcinoma, glioma, astrocyoma, medulloblastoma, craniopharyngioma, ependymoma, pinealoma, retinoblastoma, and neuroblastoma.

15. The method according to claim 14, wherein the tumor is ovarian carcinoma.

16. The method according to claim 14, wherein the tumor is lung carcinoma.

17. The method according to claim 13, wherein the tumor is non-solid tumor.

18. The method according to claim 17, wherein the non-solid tumor is selected from the group consisting of leukemia and lymphoma.

19. A method for treating an endothelial cell related condition or disorder in a subject comprising administering to a subject in need of such treatment a pharmaceutical composition comprising a therapeutically effective amount of an active agent selected from the group consisting of: (a) an isolated LEDGF/p75 polypeptide comprising the amino acid sequence as set forth in SEQ ID NO: 20, or an active analog or fragment thereof; (b) an isolated nucleic acid molecule encoding LEDGF/p75 polypeptide, the LEDGF/p75 polypeptide comprises the amino acid sequence as set forth in SEQ ID NO:20, or an active analog or fragment thereof; (c) an expression vector comprising the isolated nucleic acid molecule of (b); and (d) a host cell transfected with the expression vector of (c); further comprising a pharmaceutically acceptable carrier.

20. The method according to claim 19, wherein the isolated LEDGF/p75 polypeptide comprises the amino acid sequence as set forth in SEQ ID NO: 21 or an active analog or fragment thereof.

21. The method according to claim 19, wherein the isolated LEDGF/p75 polypeptide consists of the amino acid as set forth in SEQ ID NO: 20.

22. The method according to claim 19, wherein the isolated nucleic acid molecule encoding LEDGF/p75 polypeptide comprises the nucleotide sequence as set forth in any one of SEQ ID NO: 22 to SEQ ID NO: 24.

23. The method according to claim 19, wherein the isolated nucleic acid molecule encoding LEDGF/p75 polypeptide consists of the nucleotide sequence as set forth in SEQ ID NO: 22.

24. The method according to claim 19, wherein the condition or disorder is an inflammation.

25. The method according to claim 24, wherein the inflammation is associated with a disease or condition selected from the group consisting of: an inflammatory disease, an autoimmune disease, an infectious disease, an injury and edema.

26.-29. (canceled)


SEQUENCE LISTING (48 sequences)

SEQ ID NO: 1 - DNA, 218 bp, Artificial Sequence
  Synthetic Polynucleotide
gaccctgtttgttccttctctagctttttgtttggccctttcttcccttgatctttggttttattcgcttcctcatgctgtctttgttcagcaagagatttattcagcacttgggtgatcacggaatctccttcaccaaccaagaacatgttcttaaacttgttatacaacattgtagacttttccatgattacctgactaactttgaatcgccgtat

SEQ ID NO: 2 - DNA, 570 bp, Homo sapiens
tcttttaaaaaaaaaaaaaaaaaaggacatggcattaatactgtgaagtctgttcagggtaactcagcagcatcatcagaatacctgtatcaagtaatggcaggtgtagtctccacctatgaggctgtgttttaatttaaaaggctactgtacgctattttactttttaggtgagaagaaaacagaagcagctgagcaaccaggaagtgggtactctagggattaggaggcattgtgatggatgggtttttaaaaattgtttatccagagtcatctgcctcatgagcaatggtttctgcagaagtcgaatataaactgacatgatttcttgttctcaatgcacactgctctagtccttcaacaggcccatcagattttcaccattttgcctttgtatactttttctgtggcgtatacacagtgaaactatgtatgaaagccatttttaaaagcagtcctggcaaatggtttaaaaactggtgtgaaattgttggctttttaccacatacctgtttgttccttctctagctttttgtttggccctttcttcccttgatctttggttttatt

SEQ ID NO: 3 - DNA, 565 bp, Homo sapiens
aaccaagaacatgttcttaaacttgttatacaacattgtagacttttccatgattacctgactaactttgaatcgccgtatctgagaaaacaatatacattcatcataattgttttcctaattgattttttaaacaatttttaaatgaagtaatgacaaaattcaaagaatccacatgacttgaaatcacttacttttttcagtgtagtaatcatctctgtgtgtttctgagcttgttgcattgtgacctgaagtgaagcaagttcatccaaggcctcaatgcatctgttcacatccttcatgacaaaacagtaatttcatagctgttaattatcttaacacttaaacaatctgtttctaaagatcgtttctaaaaaaaaaaaaaaaaaaaaaaaaaaaaccttgtcggccgcctcggcccagtcgactctagactcgagcaagcttatgcatgcggccgcaattcgagctcacttggccaattcgccctatagtgagtcgtattacaattcactggccgtcgttttacaacgtcgtgactggggctggcgtaatagcgaagagg

SEQ ID NO: 4 - DNA, 635 bp, Rattus norvegicus
actctgagctaatggcgtccacatggttgtttccagtcaatggccatgcagtgttgcttcactaatggtagaggcttcttcatttctaacacattaacaaaaccatttacaaaagcagttctggcaaataactgacaagcaggacagcagcagcttaccgtatcaccacatacctgtttgttccttttctagctttttgtttggccctttcttcccttgatctttggttttatttgcttcctcatgctgtctttgttcagcaagagatttgtttagcacttgggtgatgacggaatccccttcaccaaccaaaaacatattcttaaacttgttatacaacattgtagacttctccatgataacttgactgactttgaaccgccggatctgagaaaacaacagtcacagttattttccctggttgatttacacttcctttaaaagtgaactgagaaaagacgttacatttgcaggcacttactttcttcagggttgtgatcatctctgtgtgtttctgagcttgttgcattgttacctgaagtgaagcgagttcatccagtgcctcaatgcatctgttcacatccttcatgacaaaacagcaatgtcacaactgttaattaccacagcatcttgta

SEQ ID NO: 5 - DNA, 789 bp, Bos taurus
gataatcttgtaagtgtttaatacctcatttaaaaatacagtgctgcttttttagcttcatgtttttttctaacatggtttgataagtacgtgtaaatttgagataacaaaatgttactggctattttgtatgccttgttatttgagattttgaaagtaatctctggattctatgggttgtggtaattaacagttatgaaattactgttttgtcatgaaggatgtcaacagatgtattgaggccttggatgaactggcttcacttcaggtcacaatgcaacaagctcaaaaacacacagagatgattacaacactgaaaaaagtaagtgatctttagttatgcagatttttaaaatttcctcatcaccactttgttaaataacattcagaaatcaattaggaaaattctgagcttgttttttgttttcccagatacggcgattcaaagttagtcaggttatcatggaaaagtctacaatgttgtataacaagtttaagaacatgtttttggttggtgaaggagattctgtgatcacacaagtgctgaacaaatctcttgccgaacaaagacagcatgaggaagcaaataaaaccaaagatcaagggaagaaagggccaaacaaaaagctagaaaaggagcaaacaggtatgtggtaatactttgttggaattactttttaaaaatggctttgatatttaatcttattaatgccttaacctatagtagatgatgggagtcccagaagaacttagcagagtctgctcaatcttggctaagtttttatcaag

SEQ ID NO: 6 - DNA, 499 bp, Bos taurus
atatatacaccttaaaatgtttatgactgaataatcttcaggagtgtcactgaaaaacttgcggagatataataagaccagtgattatacgttgacagctgctgacgccgactcaggaagttcatttctcctgacttttgtgtaggttagaaattttccctagtttaaatgaaaacaatagcaaccactacttctagtaactactactaagtaactactagtaatttaaacttgataaaaacttagccaagattgagcagactctgctaagttcttctgggactcccatcatctactataggttaaggcattaataagattaaatatcaaagccatttttaaaaagtaattccaacaaagtattaccacatacctgtttgctccttttctagctttttgtttggccctttcttcccttgatctttggttttatttgcttcctcatgctgtctttgttcggcaagagatttgttcagcacttgtgtgatcacagaatctc

SEQ ID NO: 7 - DNA, 3184 bp, Mus musculus
tttttttttttttttttttttttttggttttttgaaacaggatttctctgtatagccctggctgtcctggaactcactttgtagaccaggctggcctcgaactcagaaatttgcctgcctctgcctcccgagtgctgggattaaaggcgtgtgccaccacacccagctcgagtttatttttaaatatacttgagttcacacaaaggctgtacaagaccacagcatgtacatatgaagcagcagtttatccaacacatttggaacctgcagtgttgtatcttgactatagggctcaatttaatatgcatcttaaatgcactgccgtcatggcagttttattcattgacacgtacaaagcatagcataggtgtgtcagccatctatttggatagtctgctattgactgtcatcactgttcctgactccatttagatgttacctctcactagccaaccgtgttcttacagcacagtagaatacagcttgtatcaacaaacacaataccactcccaaccgtgttcttacagcacagtagaatacagcttgtatcaacaaacacaataccactcgattaacagtgtatataacatgctgggggtttaagtgacattgaaaacattggccacaatacctattggctctgatcagaaagaaagcaagcaacagcattttcagctctgcctaggatgcagcaattctggtcatatgctaactatacgttgtattttttagctaaagcaaatgagggagggagggagggagggagggaattggctggttctctaacattctgatttccctcaagagttaacaaatgtttttgttagaagtttccttcataaagctcatcataacattcttacaggtcatatacaagtactctgtgttaatggcatccccattgctactactataatatttcaaggtcaatggaggtacagtgtagccttactgagagaggttccttcatttctaacattgacaaaaaccatttagaaaagcaaccctggcaaacacctgaggcgcaagcatgacaagcatcagctgagcggacggatcaccacatacctgttggttccttctctagctttttgtttggccctttcttcccctgatctttggttttatttgcttcctcatgctgtctctgttcagcaagagatttgttcagcacttgggtgatgactgaatctccttcaccaaccaaaaacatattcttaaacttgttatataacattgtagacttttccatgataacttgactgactttgaaccgccgtatctgagaaaacagtagtcatagttattgtccctaattgattgaaactttggtgaactgagaaacgatgttagcgttgcaggcgctgagggcacttactttcttcagggttgtgatcatctctgtgtgtttctgagcttgctgcattgttacctgaagtgaagcgagttcatccagggcctctatgcatctgttcacgtccttcatgacaaaacagcaatttcataactgttaatcaccaaggcagccatggaatccacttctgaccatctcaaaagctcttaaggtggtacaaaaatggccaggtattgttccttttatcttaaaacttatatacttaagtcatgtgagaaaatatataaagctattaaagcagtgcattttaaatgagattaaacacttactagattatcaattttgagtgaatttttaatttcagcatgtatcctttgaagtcgagaatccattgatgtttctataaattgaaatatgtgaaaaacagtaaatagtttttctccacaccatgtctctgtacagtctaacatataaaagtaatggtggttataaaaattttcttcataatcttcgagaaacccatatagatgaacagtgtaaaaagttatgctaattttcttttcatatatggttccaagaagaaacatgcagcatgaaagcacaaaattacagcatgctgcatttattctgtctcgggtgacgcgatcctacttgtacttgtcacctcttaaacccctgcttcaccttgtgcctttaactgcaaggtgtttatcggagcctccagctcttagctgggctgcacaactgacaccaaataagctttaagatattagcagggttactttaattgcttaaaatgcaattgaatatggaatctgttagacttgaaaacatgttttgtaagatctaagaacatgaaatgggatttcaaaagtcatttttaaattgttcaattttaaagtcaggatcccattttgtacccctggttggcttggaacttgaggctctcccggctgtgcctccataagtgccgggattccaggtatgtgccatctcagccagctcccattaaccttctctgtaattttccatgtcttagaaagtaaaacaactaacctcgcttcttctccactttcttaacttctggcttctttccttcatctttattctgcctatcaaatgtcaacacaaatatttcaaaacactgtaactttgtgactgaggttagcagatgcccatattcccctgtgtaaaggttttaaaaaatgttccttaaaaacaatccatcagcctacaaaataggacgctgactagtttttagaactgtgtagctctacagcattaacataaagacaaaaaaattgccaaattcatctttcagtgacatacacaatggtaaaagaagatctacaaattagtaatgaattttaaagcaactttactacgtttttttaaagcatactaaagtagaccagctgtaccccacatctccaaaggacaggtaattcaagggatttagaaattggcttcaatagtaaatagacctgctttaagctactgttgtttgtttttgtttttgtttttttaagttgtagaggaggccattgttttcaaggtaaatgacatccactaatgtttacataccatttcataagtgaaaaagtaccattttactaagattaacaaaaaataaccgtttgaattagaaattttactttctagtaccttggcttggcattcaggccctattatgaaccaattaaaatagacaagaaccaaaaaaaaatacctaaaaatcaggttttttttttaaaaaaactttattttaaaattttcgttacaatttcagtttttttacttaacaagtctactgtttaatctctcaggatt

SEQ ID NO: 8 - DNA, 21 bp, Artificial Sequence
  Synthetic Oligonucleotide
guuccugauggagcuguaatt

SEQ ID NO: 9 - DNA, 21 bp, Artificial Sequence
  Synthetic Oligonucleotide
uuacagcuccaucaggaactt

SEQ ID NO: 10 - DNA, 21 bp, Artificial Sequence
  Synthetic Oligonucleotide
cagcccuguccuucagagatt

SEQ ID NO: 11 - DNA, 21 bp, Artificial Sequence
  Synthetic Oligonucleotide
ttgucgggacaggaagucucu

SEQ ID NO: 12 - DNA, 21 bp, Artificial Sequence
  Synthetic Oligonucleotide
agacagcaugaggaagcgatt

SEQ ID NO: 13 - DNA, 21 bp, Artificial Sequence
  Synthetic Oligonucleotide
ttucugucguacuccuucgcu

SEQ ID NO: 14 - DNA, 53 bp, Artificial Sequence
  Synthetic Polynucleotide
ggacatgatgaccttgttgaaagctttcaacaaggtcatcatgtccctttttg

SEQ ID NO: 15 - DNA, 57 bp, Artificial Sequence
  Synthetic Polynucleotide
aattcaaaaagggacatgatgaccttgttgaaagctttcaacaaggtcatcatgtcc

SEQ ID NO: 16 - DNA, 64 bp, Artificial Sequence
  Synthetic Polynucleotide
gatcccagacagcatgaggaagcgattcaagagatcgcttcctcatgctgtctttttttggaaa

SEQ ID NO: 17 - DNA, 64 bp, Artificial Sequence
  Synthetic Polynucleotide
agcttttccaaaaaaagacagcatgaggaagcgatctcttgaatcgcttcctcatgctgtctgg

SEQ ID NO: 18 - DNA, 60 bp, Artificial Sequence
  Synthetic Polynucleotide
gatcccgactctaaatggaggtttcaagagaagatcctccatttagagtcttttttggaa

SEQ ID NO: 19 - DNA, 63 bp, Artificial Sequence
  Synthetic Polynucleotide
agcttttccaaaaaagactctaaatggaggatcttctcttgaaagatcctccatttagagtcg

SEQ ID NO: 20 - protein, 530 aa, Homo sapiens
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQAATKQSNASSDVEVEEKETSVSKEDTDHEEKASNEDVTKAVDITTPKAARRGRKRKAEKQVETEEAGVVTTATASVNLKVSPKRGRPAATEVKIPKPRGRPKMVKQPCPSESDIITEEDKSKKKGQEEKQPKKQPKKDEEGQKEEDKPRKEPDKKEGKKEVESKRKNLAKTGVTSTSDSEEEGDDQEGEKKRKGGRNFQTAHRRNMLKGQHEKEAADRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKEQTGSKTLNGGSDAQDGNQPQHNGESNEDSKDNHEASTKKKPSSEERETEISLKDSTLDN

SEQ ID NO: 21 - protein, 528 aa, Mus musculus
MTRDFKPGDLIFAKMKGYPHWPARVDEVPDGAVKPPTNKLPIFFFGTHETAFLGPKDIFPYSENKEKYGKPNKRKGFNEGLWEIDNNPKVKFSSQQASTKQSNASSDVEVEEKETNVSKEDTDQEEKASNEDVTKAVDITTPKAARRGRKRKAEKQVDTEEAGMVTAATASNVKASPKRGRPAATEVKIPKPRGRPKVVKQPCPSDGDMVIDEDKSKKKGPEEKQPKKQLKKEEEGQKEEEKPRKEPDKKEGKKEVESKRKNLAKPGVTSTSDSEDEDDQEGEKKRKGGRNFQAAHRRNMLKGQHEKEAGDRKRKQEEQMETEQQNKDEGKKPEVKKVEKKRETSMDSRLQRIHAEIKNSLKIDNLDVNRCIEALDELASLQVTMQQAQKHTEMITTLKKIRRFKVSQVIMEKSTMLYNKFKNMFLVGEGDSVITQVLNKSLAEQRQHEEANKTKDQGKKGPNKKLEKEPTGTKSLNGGSDAQESNHPQHNGDSNEDGKDSREASSKTKPPGEEREAEISLKESTLDN

SEQ ID NO: 22 - DNA, 3377 bp, Homo sapiens
gggagccgcgcgggagcagcgcagctacggcggcggcagcggcggcgcggttgcgattccgagccgttgagacgcctctgcggcagctggtggcgcaggtggcttgcgtggacgcgggtagaggcgaccggccagcaaccgcagcgtcggcgcccgcggccccggcagcaggcgcgtcgggacgccccgaggcatcctcccccgcccgcgggcccggtagctgggcccgcgtccgccgcccgcatccccgcgccgccgcatctcctcgccgcctcccgggcttcggacccccggtctcgcccccggaaacatgactcgcgatttcaaacctggagacctcatcttcgccaagatgaaaggttatccccattggccagctcgagtagacgaagttcctgatggagctgtaaagccacccacaaacaaactacccattttcttttttggaactcatgagactgcttttttaggaccaaaggatatatttccttactcagaaaataaggaaaagtatggcaaaccaaataaaagaaaaggttttaatgaaggtttatgggagatagataacaatccaaaagtgaaattttcaagtcaacaggcagcaactaaacaatcaaatgcatcatctgatgttgaagttgaagaaaaggaaactagtgtttcaaaggaagataccgaccatgaagaaaaagccagcaatgaggatgtgactaaagcagttgacataactactccaaaagctgccagaagggggagaaagagaaaggcagaaaaacaagtagaaactgaggaggcaggagtagtgacaacagcaacagcatctgttaatctaaaagtgagtcctaaaagaggacgacctgcagctacagaagtcaagattccaaaaccaagaggcagacccaaaatggtaaaacagccctgtccttcagagagtgacatcattactgaagaggacaaaagtaagaaaaaggggcaagaggaaaaacaacctaaaaagcagcctaagaaggatgaagagggccagaaggaagaagataagccaagaaaagagccggataaaaaagaggggaagaaagaagttgaatcaaaaaggaaaaatttagctaaaacaggggttacttcaacctccgattctgaagaagaaggagatgatcaagaaggtgaaaagaagagaaaaggtgggaggaactttcagactgctcacagaaggaatatgctgaaaggccaacatgagaaagaagcagcagatcgaaaacgcaagcaagaggaacaaatggaaactgagcagcagaataaagatgaaggaaagaagccagaagttaagaaagtggagaagaagcgagaaacatcaatggattctcgacttcaaaggatacatgctgagattaaaaattcactcaaaattgataatcttgatgtgaacagatgcattgaggccttggatgaacttgcttcacttcaggtcacaatgcaacaagctcagaaacacacagagatgattactacactgaaaaaaatacggcgattcaaagttagtcaggtaatcatggaaaagtctacaatgttgtataacaagtttaagaacatgttcttggttggtgaaggagattccgtgatcacccaagtgctgaataaatctcttgctgaacaaagacagcatgaggaagcgaataaaaccaaagatcaagggaagaaagggccaaacaaaaagctagagaaggaacaaacagggtcaaagactctaaatggaggatctgatgctcaagatggtaatcagccacaacataacggggagagcaatgaagacagcaaagacaaccatgaagccagcacgaagaaaaagccatccagtgaagagagagagactgaaatatctctgaaggattctacactagataactaggttgacatacctggaatatagagaacacttgagaagtttgtaatggttttcatttgaaatagactgctggaagtttaaatttttataagcataggtttgatgttgaaaacttgttttgagggagaaaatccctttgttttaaagtaaagtaaacattatcgctaagtgtaacttgtgcagtattaacagctacattatacagtaaatgtgggataaaatccatttagaaaatgttaaactgcttttccagacatggttgtagcatattttcaattagtgtgtgtatgttaatgtgtaattgatagtagaacaaagttacatttttaaaactgctacttgtataaaccttgcctcttttcccaaatactgtgggttttgtgcatagtttttacaaaccttggatttaccagactgtcttttcactgtttgtgggttttgtagaagttacacatttttatggtagataaaatgttacttctatacaagtactcactccctttttatcaaaagttaattttaatctcacagtctacattgtgctacattatccagcttctttggaacaatgtgtgctctgtatggttttttttggtatgacaactaattaagcaactgacattgaactgagaattctacaaactataaaacattaatttttgaaggtaatttagttttgtggctgggcattcagtgaagtcttaggacttctttgcagacaactgactgggtatatataggaatgaatctggctttagggttaaatcatttaaggtccttttataggcaggcactagtaactaaaactgaaaactaagtaagtttatttttgaggaatgttgttaaaaatgtctttaggaagtcactaaaacttaattggaagaaaaaatcatgatgcttatacaataaatatgaataaatgttatataaggaaactcacctatttgaaatcatggctatattgtttttattttctagattccaaaaatacaaacactagttgttccagcattgtactttgataagtctgtacattgacgtgtatggactaaatccagggtaaaatcaatgttacaaaatttaagggtatgttaactaaaggatagcatttctaagatattttgaatattagggtcatttggcacttctcagcaagtaggatacttctcatgtttttgaaattatatgaatatggaaaaaaatggcttaagaccagcgtctctgtatgacattgtgtggttgaccctctgagataactgttttcatctacagaattgcatttttgcttttaaagaggtcttataatggaactaggaatcaccgttttgagagaacctgcatatataccagtcattatctgtttggtccttatacagttttaacttacttagatttattctagttaagccataagttcaacgtgtaaacttgttttcattaaagaatttttctatcaaaaaaaaaaaaaaaaaaaaaa

SEQ ID NO: 23 - DNA, 3396 bp, Homo sapiens
cagtgctagcgggcgccgagcgggagccgcgcgggagcagcgcagctacggcggcggcagcggcggcgcggttgcgattccgagccgttgagacgcctctgcggcagctggtggcgcaggtggcttgcgtggacgcgggtagaggcgaccggccagcaaccgcagcgtcggcgcccgcggccccggcagcaggcgcgtcgggacgccccgaggcatcctcccccgcccgcgggcccggtagctgggcccgcgtccgccgcccgcatccccgcgccgccgcatctcctcgccgcctcccgggcttcggacccccggtctcgcccccgaaacatgactcgcgatttcaaacctggagacctcatcttcgccaagatgaaaggttatccccattggccagctcgagtagacgaagttcctgatggagctgtaaagccacccacaaacaaactacccattttcttttttggaactcatgagactgcttttttaggaccaaaggatatatttccttactcagaaaataaggaaaagtatggcaaaccaaataaaagaaaaggttttaatgaaggtttatgggagatagataacaatccaaaagtgaaattttcaagtcaacaggcagcaactaaacaatcaaatgcatcatctgatgttgaagttgaagaaaaggaaactagtgtttcaaaggaagataccgaccatgaagaaaaagccagcaatgaggatgtgactaaagcagttgacataactactccaaaagctgccagaagggggagaaagagaaaggcagaaaaacaagtagaaactgaggaggcaggagtagtgacaacagcaacagcatctgttaatctaaaagtgagtcctaaaagaggacgacctgcagctacagaagtcaagattccaaaaccaagaggcagacccaaaatggtaaaacagccctgtccttcagagagtgacatcattactgaagaggacaaaagtaagaaaaaggggcaagaggaaaaacaacctaaaaagcagcctaagaaggatgaagagggccagaaggaagaagataagccaagaaaagagccggataaaaaagaggggaagaaagaagttgaatcaaaaaggaaaaatttagctaaaacaggggttacttcaacctccgattctgaagaagaaggagatgatcaagaaggtgaaaagaagagaaaaggtgggaggaactttcagactgctcacagaaggaatatgctgaaaggccaacatgagaaagaagcagcagatcgaaaacgcaagcaagaggaacaaatggaaactgagcagcagaataaagatgaaggaaagaagccagaagttaagaaagtggagaagaagcgagaaacatcaatggattctcgacttcaaaggatacatgctgagattaaaaattcactcaaaattgataatcttgatgtgaacagatgcattgaggccttggatgaacttgcttcacttcaggtcacaatgcaacaagctcagaaacacacagagatgattactacactgaaaaaaatacggcgattcaaagttagtcaggtaatcatggaaaagtctacaatgttgtataacaagtttaagaacatgttcttggttggtgaaggagattccgtgatcacccaagtgctgaataaatctcttgctgaacaaagacagcatgaggaagcgaataaaaccaaagatcaagggaagaaagggccaaacaaaaagctagagaaggaacaaacagggtcaaagactctaaatggaggatctgatgctcaagatggtaatcagccacaacataacggggagagcaatgaagacagcaaagacaaccatgaagccagcacgaagaaaaagccatccagtgaagagagagagactgaaatatctctgaaggattctacactagataactaggttgacatacctggaatatagagaacacttgagaagtttgtaatggttttcatttgaaatagactgctgaaagttttaaatttttataagcataggtttgatgttgaaaacttgttttgagggagaaaatccctttgttttaaagtaaagtaaacattatcgctaagtgtacttgtgcagtattaacagctacattatacagtaaatgtgggataaaatccatttagaaaatgttaaactgcttttccagacatggttgtagcatattttcaattagtgtgtgtatgttaatgtgtaattgatagtagaacaaagttacatttttaaaactgctacttgtataaaccttgcctcttttcccaaatactgtgggttttgtgcatagtttttacaaaccttggatttaccagactgtcttttcactgtttgtgggttttgtagaagttacacatttttatggtagataaaatgttacttctatacaagtactcactccctttttatcaaaagttaattttaatctcacagtctacattgtgctacattatccagcttctttggaacaatgtgtgctctgtatggttttttttggtatgacaactaattaagcaactgacatggaactgagaattctacaaactataaaacattaatttttgaaggtaatttagttttgtggctgggcattcagtgaagtcttaggacttctttgcagacaactgactgggtatatataggaatgaatctggctttagggttaaatcatttaaggtccttttataggcaggcactagtaactaaaactgaaaactaagtaagtttatttttgaggaatgttgttaaaaatgtctttaggaagtcactaaaacttaattggaagaaaaaatcatgatgcttatacaataaatatgaataaatgttatataaggaaactcacctatttgaaatcatggctatattgtttttattttctagattccaaaaatacaaacactagttgttccagcattgtactttgataagtctgtacattgacgtgtatggactaaatccagggtaaaatcaatgttacaaaatttaagggtatgttaactaaaggatagcatttctaagatattttgaatattagggtcatttggcacttctcagcaagtaggatacttctcatgttttgaaattatatgaatatggaaaaaaatggcttaagaccagcgtctctgtatgacattgtgtggttgaccctctgagataactgttttcatctacagaattgcatttttgcttttaaagaggtcttataatggaactaggaatcaccgttttgagagaacctgcatatataccagtcattatctgtttggtccttatacagttttaacttacttagatttattctagttaagccataagttcaacgtgtaaacttgttttcattaaagaatttttctatcaaaaaaaaaaaaaaaaaaaaaa

SEQ ID NO: 24 - DNA, 3182 bp, Mus musculus
gttgcagctcggagccgttgagacgcctctgcggcagctggtggcgcaggtggccggcgtggacgtgggcaggagcggccagcgcgccccgcagccccagcgccgccgcccggcaggcgcgtcgggacaccccgaggcatcctcccccgcccgcgggcccgccagtcccagctcgcgtctgtgctctcgcatctcctgatcgcggctgcgctgctcccccttctggtcttgggactcccggcgtcgcccctcaaacatgactcgcgatttcaaacctggagacctcatcttcgccaagatgaaaggttatcctcattggccagctcgagtagatgaagttcctgatggagctgtaaaaccacccacaaacaaactacccattttcttttttggaacccatgagactgcttttttaggaccaaaggacatatttccttattcagaaaataaggaaaagtatggcaaaccaaataaacggaaaggttttaatgaaggattgtgggagatagataacaatccgaaagtgaaattttcaagtcaacaggcatcaactaaacaatccaatgcatcgtctgatgttgaagtggaagaaaaagagactaacgtttcaaaggaagacactgatcaggaagaaaaggccagcaatgaggatgtgactaaagcagttgacataaccactccaaaagctgccaggcgaggaagaaagagaaaggctgaaaaacaagtagacactgaagaggcgggaatggtgactgcagcaaccgcttctaatgtgaaagcaagtcctaagagaggacgacctgcagctactgaagtcaagattcccaaaccaagaggcagacctaaagtggtaaagcagccttgtccttcagacggtgacatggttattgatgaagataaaagtaaaaaaaagggaccagaggagaaacaacctaaaaagcagcttaaaaaagaggaagaaggccaaaaggaagaagagaagccaagaaaagaaccagataagaaagaagggaagaaagaagttgaatctaaacggaaaaatttagctaaaccaggggttacatcaacctctgattcagaagacgaagacgatcaagagggtgaaaagaagagaaagggtggaaggaacttccaggctgctcacaggaggaacatgctaaaaggccaacatgagaaagaagctggagatcggaaacgcaagcaggaggaacaaatggaaactgagcagcagaataaagatgaaggaaagaagccagaagttaagaaagtggagaagaagcgagaaacatcaatggattctcgacttcaaaggatacatgctgaaattaaaaattcactcaaaattgataatctagacgtgaacagatgcatagaggccctggatgaactcgcttcacttcaggtaacaatgcagcaagctcagaaacacacagagatgatcacaaccctgaagaaaatacggcggttcaaagtcagtcaagttatcatggaaaagtctacaatgttatataacaagtttaagaatatgtttttggttggtgaaggagattcagtcatcacccaagtgctgaacaaatctcttgctgaacagagacagcatgaggaagcaaataaaaccaaagatcaggggaagaaagggccaaacaaaaagctagagaaggaaccaacaggtacgaagagcctaaatggtggatctgatgctcaagagagtaatcatccacagcataatggtgatagtaacgaagacggcaaagacagccgcgaggccagcagtaagacaaagccacctggagaagagagagaggctgaaatatctctgaaagagtctacactagataactaggtcggcagacttcagatgtaaagagtgtgagaagtcttgtgagtgacttcatttgaaatacttaggctgctgaaaggtttaaagttttataagcataggttttgatgtttaagtagttgggggaaaagaaaaaccctttatatttgtgtaaaagtcaattattcctaattttccgtgtgcagtattaacagctccgtcgtttagtttaagagcaagggaaacctacttagaaaggtcagcctgctttatgactgcagtgtattttcagttagtgtgtatgttaatgtgtaattggtagtggggcagttatattttaaaactgctacttgtataaatctttcccaaataccgtgggttttgtgcatagtttttacagatatggatttagcagactgtcttttcactgttatgggttttttagaagttgagcatttttatggctgataaagtgaatgttacttctaagtgctcacttcttttatcagaagtgaccctcagtccattgtgctacgttagcttgcctctttgtaataatgcgtagtctgtatgacagctagatagccaccaggttgttatagaaggtacaaatttgtttactttccaaagtaatttagttttaggactgggatttcagtggcattagaactaaagagaacagactggtatatctacatatagaaagtccacctacaatcctttatagctactagtcacaaaaagctaaaactagtttgttttgaatcttacactcactaagaaagtcactcattaaggtgtgtgttttctttttttaattagaatcaatccatgatgcttgtaagtgataaatgataaataaggaaactagcttaattgaaaacatggccatattggaatgggatttttagtttttctatcagacatattagaggatacatcagttgctctaaggtagtaaccagatagtccacacacacaaacatgaactatcactctgtcccgtctagggaggacatgtcacaggtgttctgagcgatcaggccagctgttggtttttatcaggcaggatagttcatccttaggctttggaattccattggaaagtgacttcataccagggtttttgtgcgatgtgattaaatatgttcttgatttctcagataattattttcatctatgtaaaaattacttcaataattggacttttacaacttgactgttttgagcaaacgctgtacattatatacacaccagtcttttgctttgtccttagtacatttttaacttggacaaaaattattctagttaagccataagtgtcaatgtgtaaattgttttaattaaagttttttctaccaaa

SEQ ID NO: 25 - DNA, 18 bp, Artificial Sequence
  Synthetic Oligonucleotide
ctgctcgccgctgcgctg

SEQ ID NO: 26 - DNA, 23 bp, Artificial Sequence
  Synthetic Oligonucleotide
gtgctggtgttcatgcactgcag

SEQ ID NO: 27 - DNA, 24 bp, Artificial Sequence
  Synthetic Oligonucleotide
cacacagagatgattactacactg

SEQ ID NO: 28 - DNA, 22 bp, Artificial Sequence
  Synthetic Oligonucleotide
ccatcttgagcatcagatcctc

SEQ ID NO: 29 - DNA, 21 bp, Artificial Sequence
  Synthetic Oligonucleotide
cctgttggttccttctctagc

SEQ ID NO: 30 - DNA, 21 bp, Artificial Sequence
  Synthetic Oligonucleotide
ggcggttcaaagtcagtcaag

SEQ ID NO: 31 - DNA, 21 bp, Artificial Sequence
  Synthetic Oligonucleotide
cctgtttgttccttctctagc

SEQ ID NO: 32 - DNA, 21 bp, Artificial Sequence
  Synthetic Oligonucleotide
ggcgattcaaagttagtcagg

SEQ ID NO: 33 - DNA, 24 bp, Artificial Sequence
  Synthetic Oligonucleotide
cggagtcaacggatttggtcgtat

SEQ ID NO: 34 - DNA, 24 bp, Artificial Sequence
  Synthetic Oligonucleotide
agccttctccatggtggtgaagac

SEQ ID NO: 35 - DNA, 23 bp, Artificial Sequence
  Synthetic Oligonucleotide
atgactcgcgatttcaaacctgg

SEQ ID NO: 36 - DNA, 27 bp, Artificial Sequence
  Synthetic Oligonucleotide
ctagttatctagggtagactccttcag

SEQ ID NO: 37 - DNA, 21 bp, Artificial Sequence
  Synthetic Oligonucleotide
cctgtttgttccttctctagc

SEQ ID NO: 38 - DNA, 21 bp, Artificial Sequence
  Synthetic Oligonucleotide
ggcgattcaaagttagtcagg

SEQ ID NO: 39 - DNA, 17 bp, Artificial Sequence
  Synthetic Oligonucleotide
ccgccgcagcgcccgcg

SEQ ID NO: 40 - DNA, 20 bp, Artificial Sequence
  Synthetic Oligonucleotide
gagaagaagcccagcaagtg

SEQ ID NO: 41 - DNA, 29 bp, Artificial Sequence
  Synthetic Oligonucleotide
cacttcggggaagaaaagggaggaggggg

SEQ ID NO: 42 - DNA, 47 bp, Artificial Sequence
  Synthetic Polynucleotide
gccagagccctcgtttttctcctttcttttcttccccgaagtgagag

SEQ ID NO: 43 - DNA, 468 bp, Artificial Sequence
  Synthetic Polynucleotide
ccgccgcagcgcccgcggcccggctcctctcacttcggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatg

SEQ ID NO: 44 - DNA, 21 bp, Artificial Sequence
  Synthetic Oligonucleotide
ctgtgtccagcgtagatgagc

SEQ ID NO: 45 - DNA, 21 bp, Artificial Sequence
  Synthetic Oligonucleotide
gtagacggacacacatggagg

SEQ ID NO: 46 - DNA, 8 bp, Artificial Sequence
  Synthetic Oligonucleotide
gaagggga

SEQ ID NO: 47 - DNA, 8 bp, Artificial Sequence
  Synthetic Oligonucleotide
ggaggggg

SEQ ID NO: 48 - DNA, 20 bp, Artificial Sequence
  Synthetic Oligonucleotide
gaaggggagggaggaggggg